(19) United States
(12) Patent Application Publication
(10) Pub. No.: US 2024/0025829 A1
(43) Pub. Date: Jan. 25, 2024

(54) INTEGRATED PROCESS FOR THE PARALLEL PRODUCTION OF ALKALI METAL METHOXIDES

(71) Applicant: BASF SE, Ludwigshafen am Rhein (DE)

(72) Inventors: Wolf-Steffen WEISSKER, Ludwigshafen am Rhein (DE); Josef GUTH, Freinsheim (DE); Kai HOFEN, Ludwigshafen am Rhein (DE); Holger FRIEDRICH, Ludwigshafen am Rhein (DE)

(21) Appl. No.: 18/039,501

(22) PCT Filed: Dec. 3, 2021
(86) PCT No.: PCT/EP2021/084140

§ 371 Date: May 31, 2023

(30) Foreign Application Priority Data
Dec. 4, 2020 (EP) 20211938.4

(51) Int. Cl. C07C 29/70; B01D 3/14; B01D 3/00; C07C 31/30

(52) U.S. Cl. CPC C07C 29/70; B01D 3/143; B01D 3/009; C07C 31/30

(57) ABSTRACT

The present invention relates to an integrated process for simultaneously preparing at least two mixtures comprising alkali metal methoxide and methanol in at least two parallel reactive distillation columns, wherein one rectification column is used for providing a methanol stream which is then used as methanol source for the reactive distillation columns, and wherein the top streams of said reactive distillation columns are used as methanol source for said rectification column.

The present invention relates to a new and highly advantageous process for simultaneously preparing alkali metal methoxides in two or more parallel reactive distillation columns. Further, the present invention relates to a chemical production unit for carrying out this process.

In the prior art, processes are described wherein a mixture comprising an alkali metal alkoxide and methanol is prepared in a reactive distillation column from a methanol stream and an aqueous stream which comprises a dissolved alkali metal hydroxide. According to these processes, the methanol stream fed into the reactive distillation column is prepared by separating methanol from water in a distillation column upstream of the reactive distillation column and using the respectively obtained methanol to the reactive distillation column. In this respect, reference is made, for example, to US 2002/0183566 A1, US 2008/0296786 A1, or WO 2013/168113 A1.

However, according to the teaching of these prior art documents, only one specific mixture comprising alkali metal methoxide and methanol can be produced. Consequently, for the simultaneous production of e.g. two different mixtures comprising alkali metal methoxide and methanol, such as two mixtures comprising the same alkali metal methoxide but differing in the alkali metal methoxide concentration or two mixtures differing in the alkali metal, two complete separate plants would be necessary, each of the two plants comprising a distillation column for providing the methanol stream and a reactive distillation column. Alternatively, if only one plant is used, it is necessary to produce a first mixture in a first run, stop the first process, re-adjust the production parameters, and start the production of the second mixture. Both alternatives, however, are economically highly disadvantageous since according to the first alternative which would allow for a simultaneous production of two or more different mixtures comprising alkali metal methoxide and methanol, two or more entire plants would be required, leading e.g. to enormous equipment costs; according to the second alternative, the sequential production of the two different mixtures renders the plant disadvantageous in terms of flexibly reacting to changing market demands.

Therefore, it was an object of the present invention to provide an economically advantageous process for simultaneously preparing two or more mixtures comprising alkali metal hydroxide and methanol. It was a further object of the present invention to provide a process for preparing two or more mixtures comprising alkali metal hydroxide and methanol which allows for an easy adjustment of the amount of the mixtures prepared depending on the respective demand of the market.

Surprisingly, it was found that these objects can be solved by a process according to which a single distillation column is employed for generating a methanol stream which is then used, after a suitable dividing into two or more substreams, as a methanol source for two or more parallel downstream reactive distillation columns in which two or more different mixtures comprising alkali metal methoxide and methanol are simultaneously prepared.

Therefore, the present invention relates to an integrated process for simultaneously preparing n mixtures P(i) comprising alkali metal methoxide and methanol, comprising
providing n reactive distillation columns K(i);
providing n aqueous liquid streams H(i), a given stream H(i) comprising a dissolved alkali metal hydroxide A(i)OH, wherein n is an integer with n≥2 and i=1 . . . n; and
providing a rectification column D;
the process further comprising
(a) providing a stream G comprising methanol;
(b) dividing the stream G into n streams G(i), each stream G(i) having the same composition as G;
(c) preparing the one or more alkali metal methoxides comprising
   feeding each stream G(i) into the lower part of a respective reactive distillation column K(i), and feeding the aqueous liquid stream H(i) comprising the dissolved alkali metal hydroxide A(i)OH into the upper part of said reactive distillation column K(i); and
   subjecting G(i) and H(i) in each K(i) to reactive distillation conditions, obtaining n top streams W(i) comprising methanol and water; and obtaining n bottoms streams P(i) comprising alkali metal methoxide A(i)OMe and methanol;
(d) feeding each stream W(i) into the lower part of the rectification column D, and feeding a stream M comprising methanol into the rectification column D; and subjecting the n streams W(i) and M in D to distillation conditions, obtaining the stream G according to (a) as a top stream.

Compared to the teaching of the prior art, the process according to the present invention allows for significantly saving apparatus costs since for all reactive distillation columns, only one rectification column is necessary; yet further, the process according to the present invention allows for very flexibly reacting to market demands in terms of the supply of different mixtures comprising alkali metal methoxide and methanol since the respective amounts of different mixtures can be easily adjusted by suitably dividing the methanol stream obtained from the upstream single distillation column.

It is preferred that, in the process according to the present invention, n is in the range of from 2 to 10, more preferably in the range of from 2 to 5, such as 2, 3, 4 or 5, more preferably 2 or 3, more preferably 2.

As to the alkali metal hydroxide A(i)OH, it is preferred that each alkali metal hydroxide A(i)OH is selected from the group consisting of lithium hydroxide, sodium hydroxide and potassium hydroxide, more preferably from the group consisting of sodium hydroxide and potassium hydroxide. According to the process of the present invention, it is conceivable that at least 2 alkali metal hydroxides A(i)OH are the same; in this case, the present invention allow, for example, to prepare a first mixture P(i) comprising a first alkali metal hydroxides A(i)OH and exhibiting a first alkali metal hydroxide concentration and a second mixture P(i) comprising said first alkali metal hydroxide A(i)OH and exhibiting a second alkali metal hydroxide concentration which is different from the first alkali metal hydroxide concentration; thus, according to this conceivable process, it is possible to prepare 2 mixtures P(i) exhibiting different concentration specifications which may demanded by the market. Preferably, at least 2 of the alkali metal hydroxides A(i)OH are different from each other, wherein more preferably, in particular if n is 2 or 3, preferably 2, all alkali metal hydroxides A(i)OH are different from each other.

A given aqueous liquid stream H(i) comprises an alkali metal hydroxide A(i)OH dissolved in water. It may be preferred that at least one of these streams H(i) may comprise, in addition to water and the dissolved alkali metal hydroxide A(i)OH, a certain amount of methanol. Preferably, a given aqueous liquid stream H(i) essentially consists of an alkali metal hydroxide A(i)OH dissolved in water.

As mentioned above, it is preferred that according to the present invention, it is preferred that 2 mixtures P(i) are simultaneously prepared. Therefore, the present invention preferably relates to an integrated process for simultaneously preparing 2 mixtures P(i), wherein the mixture P(1) comprises A(1)OMe, more preferably sodium methoxide, and methanol and the mixture P(2) comprises A(2)OMe, more preferably potassium methoxide, and methanol, the process comprising
(a) providing a stream G comprising methanol;
(b) dividing the stream G into at least two streams G(1) and G(2), G(1) and G(2) having the same composition as G;
(c.1) preparing A(1)OMe, comprising
   (c.1.1) feeding the stream G(1) into the lower part of a reactive distillation column K(1), and feeding a liquid stream H(1), preferably an aqueous liquid stream H(1), H(1) comprising dissolved A(1)OH, more preferably sodium hydroxide, into the upper part of the reactive distillation column K(1);
   (c.1.2) subjecting G(1) and H(1) in K(1) to reactive distillation conditions, obtaining a top stream W(1) comprising methanol and water; and obtaining a bottoms stream P(1) comprising A(1)OMe and methanol;
(c.2) preparing A(2)OMe, comprising
   (c.2.1) feeding the stream G(2) into the lower part of a reactive distillation column K(2), K(2) being arranged in parallel with K(1), and feeding a liquid stream H(2), preferably an aqueous liquid stream H(2), H(2) comprising dissolved A(2)OH, more preferably potassium hydroxide, into the upper part of the reactive distillation column K(2);
   (c.2.2) subjecting G(2) and H(2) in K(2) to reactive distillation conditions, obtaining a top stream W(2) comprising methanol and water; and obtaining a bottoms stream P(2) comprising A(2)OMe and methanol;
(d.1) feeding W(1) and W(2) into the lower part of a rectification column D, and feeding a stream M comprising methanol into the rectification column D;
(d.2) subjecting W(1), W(2) and M in D to distillation conditions, obtaining the stream G according to (a) as a top stream.

Preferably, the streams W(1) and W(2) are fed as gas streams into the rectification column D. Generally, these streams can be fed into the rectification column D, independently from each other, at any suitable position. Preferably, these streams are fed, independently from each other, at a position between the bottoms and the 15^{th} theoretical stage, more preferably between the bottoms and the 10^{th} theoretical stage, more preferably between the bottoms and the 8^{th} theoretical stage of the rectification column D. According to the present invention, it is possible to feed the streams W(1) and W(2) into D as separate streams, or to suitably combine the streams W(1) and W(2) and feed the respective combined stream W into D.

As to the rectification column D, it is preferred that it has from 20 to 100, more preferably from 30 to 80, more preferably from 40 to 60 theoretical stages. Conceivable preferred ranges are, for example, from 40 to 50 or from 45 to 55 or from 50 to 60. Generally, the pressure at the top of the rectification column D can be chosen freely within a wide range with the proviso that the desired separation task is fulfilled. Preferably, the rectification column D is operated at a pressure at the top of D in the range of from 0.5 to 10 bar(abs), more preferably in the range of from 0.75 to 6 bar(abs), more preferably in the range of from 1 to 5 bar(abs). Conceivable preferred ranges are, for example, from 1 to 3 bar(abs) or from 2 to 4 bar(abs) or from 3 to 5 bar(abs).

According to the present invention, a stream M comprising methanol is fed into the distillation column D. This stream M, also referred to as fresh methanol stream M, is fed into D in order provide sufficient methanol for the overall process, in particular to compensate the loss of methanol removed from the process via the mixtures P(i). Generally, there are no specific requirements as far as the methanol content of M is concerned, and the skilled person will be in the position to choose suitable methanol streams M. Preferably, however, it is preferred that the stream M comprises only a low amount of water. Therefore, it is further preferred that from 99 to 100 weight-%, more preferably from 99.5 to 100 weight-%, more preferably from 99.9 to 100 weight-% of the stream M consist of methanol and optionally water, wherein the amount of water comprised in the stream M is preferably at most 2000 weight-ppm, more preferably at most 1500 weight-ppm, more preferably at most 1000 weight-ppm, such as at most 750 weight-ppm or at most 500 weight-ppm or at most 250 weight-ppm.

Generally, the stream M can be fed into the rectification column D at any suitable position. Preferably, the stream M is fed to the upper part of D, more preferably at least 2, 3 or 4 theoretical stages from the top of D, more preferably at least 4 theoretical stages from the top of D, more preferably between the 4^{th} and the 20^{th} theoretical stage from the top of D, more preferably between the 6^{th} and the 15^{th} theoretical stage from the top of D, such as between the 6^{th} and the 10^{th} theoretical stage or between to 8^{th} and the 12^{th} theoretical stage or between the 10^{th} and the 14^{th} theoretical stage or between to 12^{th} and the 15^{th} theoretical stage. As far as the temperature of the stream M is concerned at which the stream M is fed into D, it is preferred that the temperature is in the range of from ambient temperature up to the boiling point of methanol at the column pressure of D; more preferably the temperature is ambient temperature.

Generally, it may be conceivable that the rectification column D is operated without reflux, i.e. at a reflux ratio of 0:1. However, it is particularly preferred that the rectification column D is operated with reflux. Preferably, the rectification column D is operated at a reflux ratio of at least 0.5:1, preferably in the range of from 0.55:1 to 1.4:1, more preferably in the range of from 0.6:1 to 1.4:1. Suitable preferred ranges are, for example, from 0.6:1 to 1.0:1 or from 0.8:1 to 1.2:1 or from 1.0:1 to 1.4:1.

According to the present invention, it is possible that the rectification column D is operated without top vapor recompression. This is, for example, illustrated by the schematic overview in FIGS. 1, 2 and 3 showing a process according to the present invention with reflux. In case the rectification column D is operated without top vapor recompression, it is preferred that realizing the desired reflux ratio comprises removing, in addition to G, a top stream T(2) from the rectification column, passing said stream T(2) through a condenser V(4), obtaining a liquid stream T(3) and a waste gas stream T(2w), and feeding the liquid stream T(3) back into the top of the rectification column D; as far as the process feature “removing, in addition to G, a top stream T(2) from the rectification column” is concerned, it covers the possibility to remove 2 separate streams G and T(2) from the top of D as well as removing one single stream from the top of D and divide said single stream into the 2 streams G and T(2). Alternatively, in case the rectification column D is operated without top vapor recompression, it may be preferred that realizing the desired reflux ratio comprises removing, in addition to G, a top stream T(2) from the rectification column, passing said stream T(2) through a condenser V(4), obtaining a liquid stream T(2l) and a gas stream T(2g); passing the gas stream T(2g) through a condenser V(5), obtaining a liquid stream T(2gl) and a waste gas stream T(2w); and combining the liquid streams T(2l) and (T2gl), for example in a condensate drum, obtaining a combined liquid stream which is then fed as the stream T(3) back into the top of the rectification column D; again, as far as the process feature “removing, in addition to G, a top stream T(2) from the rectification column” is concerned, it covers the possibility to remove 2 separate streams G and T(2) from the top of D as well as removing one single stream from the top of D and divide said single stream into the 2 streams G and T(2). Certainly, the skilled person may also realize, if need be, said reflux ratio by using more than the 2 condensers V(4) and V(5) mentioned above. As far as the waste gas stream T(2w) is concerned, it is preferred that it essentially consists of oxygen, nitrogen, carbon dioxide and methanol, wherein the amount of methanol in T(2w) is preferably in the range of from 2 to 80 weight-%, more preferably in the range of from 10 to 30 weight-% based on the total weight of T(2w).

In particular in view of overall energy consumption topics, it may be preferred that the rectification column D is operated with top vapor recompression. Reference is made, for example, to the schematic overview in FIGS. 4, 5 and 6 showing a process according to the present invention with reflux. In case the rectification column is operated with top vapor recompression, it is preferred that realizing the reflux ratio comprises
(i) removing, in addition to G, a top stream T(2) from the rectification column D, passing said stream T(2) through a condenser V(4), obtaining a liquid stream and a waste gas stream T(2w); as far as the process feature “removing, in addition to G, a top stream T(2) from the rectification column” is concerned, it covers the possibility to remove 2 separate streams G and T(2) from the top of D as well as removing one single stream from the top of D and divide said single stream into the 2 streams G and T(2);
(ii) removing, in addition to G and T(2), a further top stream T(1) from the rectification column D, passing said stream T(2) through a compressor C(3), passing the thus compressed stream through a reboiler V(6), obtaining a liquid stream, wherein the reboiler V(6) is preferably a reboiler of the rectification column D; as far as the process feature “removing, in addition to G and T(2), a further top stream T(1) from the rectification column”, is concerned, it covers the possibility to remove 3 separate streams G and T(2) from the top of D as well as removing one single stream from the top of D and divide said single stream into the 3 streams G and T(2) and T(1) as well as removing 2 separate streams from the top of D and suitably divide these 2 streams into the streams G, T(2) and T(1);
(iii) feeding the liquid streams obtained according to (i) and (ii) into the top of the rectification column D.

Certainly, as far as step (i) above is concerned, the skilled person may also realize, if need be, said reflux ratio by using more than one condenser V(4) mentioned above. A preferred realization of the use of 2 condensers, V(4) and V(5), is described hereinunder.

When the rectification column is operated with top vapor recompression, it is also preferred that realizing the reflux ratio comprises
(i) removing, in addition to G, a top stream T(2) from the rectification column D, passing said stream T(2) through a condenser V(4), obtaining a liquid stream T(2l) and a gas stream T(2g); passing the gas stream T(2g) through a condenser V(5), obtaining a liquid stream T(2gl) and a waste gas stream T(2w); and combining the liquid streams T(2l) and (T2gl), for example in a condensate drum, obtaining a combined liquid stream T(2cl); as far as the process feature “removing, in addition to G, a top stream T(2) from the rectification column” is concerned, it covers the possibility to remove 2 separate streams G and T(2) from the top of D as well as removing one single stream from the top of D and divide said single stream into the 2 streams G and T(2);
(ii) removing, in addition to G and T(2), a further top stream T(1) from the rectification column D, passing said stream T(2) through a compressor C(3), passing the thus compressed stream through a reboiler V(6), obtaining a liquid stream, wherein the reboiler V(6) is preferably a reboiler of the rectification column D; as far as the process feature “removing, in addition to G and T(2), a further top stream T(1) from the rectification column”, is concerned, it covers the possibility to remove 3 separate streams G and T(2) from the top of D as well as removing one single stream from the top of D and divide said single stream into the 3 streams G and T(2) and T(1) as well as removing 2 separate streams from the top of D and suitably divide these 2 streams into the streams G, T(2) and T(1); according to process of the invention it may be preferred that the liquid stream obtained from the reboiler V(6) is first fed to a condensate from which a gas stream T(1g) and a liquid stream T(11) are removed, wherein the gas stream T(1g) may contain, for example, one or more inerts; this stream T(1g) can be fed, for example, into the condenser (V4) or V(5), preferably V(4), wherein the liquid stream T(11) is preferably combined with the liquid streams T(2l) and (T2gl), for example in a condensate drum, preferably in the condensate drum described in step (i) above; reference is made to step (iii) below;
(iii) feeding the combined liquid stream obtained according to (i) and the liquid stream obtained according to (ii) into the top of the rectification column D.

Certainly, as far as step (i) above is concerned, the skilled person may also realize, if need be, said reflux ratio by using more than the 2 condensers V(4) and V(5).

As mentioned above, it is preferred that (ii) further comprises feeding the liquid stream obtained from the reboiler V(6) to a condensate drum, wherein from said condensate drum, a gas stream T(1g) and a liquid stream T(11) are removed, said gas stream T(1g) being fed into the condenser V(4) and said liquid stream T(11) the liquid stream obtained according to (ii), wherein prior to being fed into the top of the rectification column D according to (iii), the liquid stream is more preferably depressurized. Preferably, (iii) comprises combining the liquid streams obtained according to (i) and (ii) to obtain a liquid stream T(3) and feeding the stream T(3) into the top of the rectification column D.

As far as the waste gas stream T(2w) is concerned, it is preferred that it essentially consists of oxygen, nitrogen, carbon dioxide and methanol, wherein the amount of methanol in T(2w) is preferably in the range of from 2 to 80 weight-%, more preferably in the range of from 10 to 30 weight-% based on the total weight of T(2w).

According to (ii), it is preferred that the reboiler V(6) is an intermediate reboiler of the rectification column D as indicated, for example, in FIGS. 4, 5 and 6. Generally, it would also be conceivable that the reboiler V(6) downstream the compressor C(3) is not used as intermediate reboiler but arranged so as to provide heat to the bottom of the column D. In this case, it would be advantageous to equip the column D with a further bottom reboiler instead of V(3), or together with a smaller dimensioned reboiler V(3), wherein said additional reboiler preferably would be dimensioned smaller than V(3) or V(6), and wherein this further reboiler would be used mainly for starting up the rectification column D. During regular operation mode of the column D, it would be conceivable to switch off said additional reboiler.

With respect to the stream G provided according to (a) obtained from distillation according to (d.2), it is preferred that said stream G comprises methanol and water, wherein more preferably from 99.95 to 100 weight-% of G consist of methanol and water, and wherein the water content of G is at most 200 weight-ppm, more preferably at most 150 weight-ppm, more preferably at most 100 weight-ppm, wherein more preferably, said water content is in the range of from 5 to 100 weight-ppm, more preferably in the range of from 10 to 100 weight-ppm, more preferably in the range of from 15 to 100 weight-ppm.

According to (b), it is preferred that the stream G is divided into the two streams G(1) and G(2), wherein the stream G has a mass flow rate f(G), the stream G(1) has a mass flow rate f(G(1)) and the stream G(2) has a mass flow rate f(G(2)), wherein f(G)=f(G(1))+f(G(2)). Generally, the stream G can be divided by any conceivable method. Preferably dividing according to (b) comprises passing the stream G into a stream dividing device S, said device more preferably comprising a pipe junction. In context, it is noted that the term “the stream is divided into two streams” refers to a method according to which the streams obtained from said dividing have the same chemical composition as the stream G. As far as the ratios f(G(1))/f(G) and f(G(2))/f(G) are concerned, the present invention allows for a flexible adjusting of said ratios in that the individual flow rates f(G(1)) and f(G(2)) can be chosen depending on the desired amount of A(1)OMe, preferably sodium methoxide, to be obtained according to (c.1.2) relative to the desired amount of A(2)OMe, preferably potassium methoxide, to be obtained according to (c.2.2).

Prior to dividing according to (b), the stream G can be passed through a compressor C, thereby realizing a pressure increase of G. Preferably, the pressure is suitably increased so that the pressure of the streams after dividing is adapted to the desired pressure when the streams are fed into the reactive distillation columns K(i) and ultimately, via the streams W(i), back into D. Preferably, said pressure increase is in the range of from 0.1 to 0.8 bar(abs), more preferably in the range of from 0.15 to 0.6 bar(abs), more preferably in the range of from 0.2 to 0.4 bar(abs). According to this embodiment of the present invention, it is preferred that the dividing according to (b) comprises passing the compressed stream G into a stream dividing device S, said device preferably comprising a pipe junction and at least one control device allowing for adjusting the ratio f(G(1))/f(G(2)), wherein said at least one control device is located downstream of said pipe junction. At least one of these control devices is located either in the stream G(1) or in the stream G(2) or in both streams G(1) and G(2), and it is preferred that the at least one control device preferably a control valve.

Preferably, according to the present invention, the pressure increase mentioned above is realized not by compressing the stream G prior to, but after dividing. Thus, according to the present invention, it is preferred that prior to be fed into the reactive distillation column K(1), the stream G(1) is passed through a compressor C(1), thereby realizing a pressure increase of G(1) in the range of from 0.1 to 0.8 bar(abs), preferably in the range of from 0.15 to 0.6 bar(abs), more preferably in the range of from 0.2 to 0.4 bar(abs). Certainly, said compression of G(1) can be combined with a pre-compression of the stream G prior to dividing; however, it is preferred that this compressing of G(1) is performed with no compression of G being performed prior to dividing according to (b). Consequently, it is also preferred that prior to be fed into the reactive distillation column K(2), the stream G(2) is passed through a compressor C(2), thereby realizing a pressure increase of G(2) in the range of from 0.1 to 0.8 bar(abs), preferably in the range of from 0.15 to 0.6 bar(abs), more preferably in the range of from 0.2 to 0.4 bar(abs). Certainly, said compression of G(2) can be combined with a pre-compression of the stream G prior to dividing; however, it is preferred that this compressing of G(2) is performed with no compression of G being performed prior to dividing according to (b).

As far as the stream H(1) is concerned, it is preferred that from 99 to 100 weight-%, more preferably from 99.5 to 100 weight-%, more preferably from 99.9 to 100 weight-% of the stream H(1) consist of A(1)OH and water, wherein more preferably from 37.5 to 58 weight-%, more preferably from 40 to 55 weight-%, more preferably from 42.5 to 52 weight-% of the stream H(1) consist of A(1)OH, preferably sodium hydroxide. Preferably the stream H(1) is fed into the reactive distillation column K(1) at a temperature of H(1) in the range of from ambient temperature to its boiling temperature, more preferably in the range of from 50 to 80° C. such as from 50 to 60° C. or from 60 to 70° C. or from 70 to 80° C. Heating of the stream H(1) to this temperature may be accomplished with any suitable means such as a heat exchanger. It is preferred that the stream H(1) is fed into the top of the reactive distillation column K(1), more preferably to the first theoretical stage from the top.

As to the reactive distillation column K(1), it is preferred that said column has from 5 to 50, more preferably from 10 to 40, more preferably from 15 to 30 theoretical stages, such as from 15 to 20 or from 20 to 25 or from 25 to 30 theoretical stages. Generally, the stream G(1) can be fed at any suitable position into K(1); preferably, G(1) is fed into the reactive distillation column K(1) at a position between the bottoms and the 5^{th} theoretical stage, more preferably between the bottoms and the 3^{rd} theoretical stage, more preferably between the bottoms and the 2^{nd} theoretical stage of the reactive distillation column K(1). Preferably, the reactive distillation column K(1) is operated at a pressure at the top in the range of from 0.5 to 10 bar(abs), more preferably in the range of from 1 to 6 bar(abs), more preferably in the range of from 1 to 5 bar(abs). Suitable preferred ranges are, for example, from 1 to 3 bar(abs) or from 2 to 4 bar(abs) of from 3 to 5 bar(abs). While it is generally possible to operate the reactive distillation column K(1) with reflux, it is preferred that the reactive distillation column K(1) is operated at a reflux ratio of 0:1.

As far as the stream W(1) is concerned which is obtained from the top of K(1), it is preferred that from 99 to 100 weight-%, more preferably from 99.5 to 100 weight-%, more preferably from 99.9 to 100 weight-% of W(1) consist of methanol and water. More preferably, from 1 to 10 weight-%, more preferably from 2 to 8 weight-%, more preferably from 4 to 7 weight-%, more preferably from 5 to 6 weight-% of the stream W(1) consist of water.

As far as the mixture P(1) is concerned, it is preferred that from 99 to 100 weight-%, more preferably from 99.5 to 100 weight-%, more preferably from 99.9 to 100 weight-% of the stream P(1) consist of A(1)OMe, preferably sodium methoxide, and methanol. More preferably, from 10 to 50 weight-%, more preferably from 20 to 40 weight-%, more preferably from 25 to 35 weight-% of the stream P(1) consist of A(1)OMe, preferably sodium methoxide. More preferably, at most 5000 weight-ppm, more preferably at most 2000 weight-ppm, more preferably at most 1000 weight-ppm of the stream P(1) consist of water. Conceivable maximum water contents may include, for example, 750 weight-ppm or 500 weight-ppm or 250 weight-ppm. Preferably, the concentration of A(1)OMe, preferably sodium methoxide in the stream P(1) are realized by the skilled person by operating the reactive distillation column K(1) at a respective reboiler duty.

According to the present invention, it is preferred that the top of the reactive distillation column K(1) is equipped with a suitable droplet separating device D(1), preferably a demister. Thus, the process preferably comprises separating droplets comprising A(1)OH, preferably sodium hydroxide, from the vapor stream in the top of K(1). It is further preferred that, in particular for cleaning purposes, said demister is suitably treated with a suitable stream M(1). A preferred treating may comprise, preferably consist of at least temporarily spraying the demister with the stream M(1). Regarding the chemical composition of M(1), it is especially preferred that M(1) comprises methanol, wherein it is more preferred that M(1) is branched from a condensed top stream removed from the rectification column D, for example one of the streams described above, or being a fresh methanol stream, for example a stream branched from the stream M described above.

As far as the stream H(2) is concerned, it is preferred that from 99 to 100 weight-%, more preferably from 99.5 to 100 weight-%, more preferably from 99.9 to 100 weight-% of the stream H(2) consist of A(2)OH, preferably potassium hydroxide, and water, wherein more preferably 30 to 55 weight-%, more preferably from 40 to 52.5 weight-%, more preferably from 45 to 50 weight-% of the stream H(2) consist of A(2)OH, preferably potassium hydroxide. Preferably the stream H(2) is fed into the reactive distillation column K(2) at a temperature of H(2) in the range of from ambient temperature to its boiling temperature, more preferably in the range of from 50 to 80° C. such as from 50 to 60° C. or from 60 to 70° C. or from 70 to 80° C. Heating of the stream H(2) to this temperature may be accomplished with any suitable means such as a heat exchanger. It is preferred that the stream H(2) is fed into the top of the reactive distillation column K(2), more preferably to the first theoretical stage from the top.

As to the reactive distillation column K(2), it is preferred that said column has from 5 to 50, more preferably from 10 to 40, more preferably from 15 to 30 theoretical stages, such as from 15 to 20 or from 20 to 25 or from 25 to 30 theoretical stages. Generally, the stream G(2) can be fed at any suitable position into K(2); preferably, G(2) is fed into the reactive distillation column K(2) at a position between the bottoms and the 5^{th} theoretical stage, more preferably between the bottoms and the 3^{rd} theoretical stage, more preferably between the bottoms and the 2^{nd} theoretical stage of the reactive distillation column K(2). Preferably, the reactive distillation column K(2) is operated at a pressure at the top in the range of from 0.5 to 10 bar(abs), more preferably in the range of from 1 to 6 bar(abs), more preferably in the range of from 1 to 5 bar(abs). Suitable preferred ranges are, for example, from 1 to 3 bar(abs) or from 2 to 4 bar(abs) of from 3 to 5 bar(abs). While it is generally possible to operate the reactive distillation column K(2) with reflux, it is preferred that the reactive distillation column K(2) is operated at a reflux ratio of 0:1.

As far as the stream W(2) is concerned which is obtained from the top of K(2), it is preferred that from 99 to 100 weight-%, more preferably from 99.5 to 100 weight-%, more preferably from 99.9 to 100 weight-% of W(2) consist of methanol and water. More preferably, from 1 to 15 weight-%, more preferably from 2 to 12 weight-%, more preferably from 6 to 10 weight-% of the stream W(2) consist of water.

As far as the mixture P(2) is concerned, it is preferred that from 99 to 100 weight-%, more preferably from 99.5 to 100 weight-%, more preferably from 99.9 to 100 weight-% of the stream P(2) consist of A(2)OMe, preferably potassium methoxide, and methanol. More preferably, from 10 to 50 weight-%, more preferably from 20 to 40 weight-%, more preferably from 25 to 35 weight % of the stream P(2) consist of A(2)OMe, preferably potassium methoxide. More preferably, at most 5000 weight-ppm, more preferably at most 2000 weight-ppm, more preferably at most 1000 weight-ppm of the stream P(2) consist of water. Conceivable maximum water contents may include, for example, 750 weight-ppm or 500 weight-ppm or 250 weight-ppm. Preferably, the concentration of A(2)OM, preferably potassium methoxide in the stream P(2) are realized by the skilled person by operating the reactive distillation column K(2) at a respective reboiler duty.

According to the present invention, it is preferred that the top of the reactive distillation column K(2) is equipped with a suitable droplet separating device D(2), preferably a demister. Thus, the process preferably comprises separating droplets comprising A(2)OH, preferably potassium hydroxide, from the vapor stream in the top of K(2). It is further preferred that, in particular for cleaning purposes, said demister is suitably treated with a suitable stream M(2). A preferred treating may comprise, preferably consist of at least temporarily spraying the demister with the stream M(2). Regarding the chemical composition of M(2), it is especially preferred that M(2) comprises methanol, wherein it is more preferred that M(2) is branched from a condensed top stream removed from the rectification column D, for example one of the streams described above, or being a fresh methanol stream, for example a stream branched from the stream M described above.

In the above, it was described that according to the present invention, either the stream G, prior to dividing, is passed through a compressor C, and/or after dividing, the stream G(1) is passed through a compressor C(1) and the stream G(2) is passed through a compressor C(2), the latter being preferred. According to the present invention, it is also possible that either in addition to at least one of the above alternatives or, preferably as the sole respective compression, prior to being fed into the rectification column D, the stream W(1) is passed through a compressor C(1), thereby realizing a pressure increase of W(1) preferably in the range of from 0.1 to 0.8 bar(abs), more preferably in the range of from 0.15 to 0.6 bar(abs), more preferably in the range of from 0.2 to 0.4 bar(abs), and prior to being fed into the rectification column D, the stream W(2) is passed through a compressor C(2), thereby realizing a pressure increase of W(2) in the range of from 0.1 to 0.8 bar(abs), more preferably in the range of from 0.15 to 0.6 bar(abs), more preferably in the range of from 0.2 to 0.4 bar(abs). According to this embodiment of the present invention, it is also possible to suitably combine the streams W(1) and W(2), prior to being passed through a compressor, in a combining device to obtain a respective combined stream W, and pass said combined stream W, prior to being fed into D, through a compressor, thereby realizing a pressure increase of W(1) preferably in the range of from 0.1 to 0.8 bar(abs), more preferably in the range of from 0.15 to 0.6 bar(abs), more preferably in the range of from 0.2 to 0.4 bar(abs). Said combining device preferably comprises a pipe junction and at least one control device, preferably a control valve.

As far as the integrated process of the present invention is concerned, it is noted that for simultaneously preparing, in addition to the 2 mixtures P(1) and P(2) as described above in detail, a 3^{rd} mixture P(3), and/or a 4^{th} mixture P(4), and/or a 5^{th} mixture P(5) etc., the skilled person, based on his general knowledge, will be in the position to derive from said details above in a straight-forward manner also any detail for operating the respective additional compressors, reactive distillation columns, and the like. In particular, according to the present invention, it is preferred that the process of the present invention is an integrated process for simultaneously preparing three mixtures P(1), P(2) and P(3), wherein the mixture P(1) comprises sodium methoxide and methanol, the mixture P(2) comprises potassium methoxide and methanol, and the mixture P(3) comprises lithium methoxide and methanol, the process comprising
(a) providing a stream G comprising methanol;
(b) dividing the stream G into three streams G(1), G(2) and G(3), G(1), G(2) and G(3) having the same composition as G;
(c.1) preparing sodium methoxide comprising
   (c.1.1) feeding the stream G(1) into the lower part of a reactive distillation column K(1), and feeding an aqueous liquid stream H(1) comprising dissolved sodium hydroxide, into the upper part of the reactive distillation column K(1);
   (c.1.2) subjecting G(1) and H(1) in K(1) to reactive distillation conditions, obtaining a top stream W(1) comprising methanol and water; and obtaining a bottoms stream P(1) comprising sodium methoxide and methanol;
(c.2) preparing potassium methoxide comprising
   (c.2.1) feeding the stream G(2) into the lower part of a reactive distillation column K(2), K(2) being arranged in parallel with K(1), and feeding an aqueous liquid stream H(2) comprising dissolved potassium hydroxide, into the upper part of the reactive distillation column K(2);
   (c.2.2) subjecting G(2) and H(2) in K(2) to reactive distillation conditions, obtaining a top stream W(2) comprising methanol and water; and obtaining a bottoms stream P(2) comprising potassium methoxide and methanol;
(c.3) preparing lithium methoxide comprising
   (c.3.1) feeding the stream G(3) into the lower part of a reactive distillation column K(3), K(3) being arranged in parallel with K(1) and K(2), and feeding an aqueous liquid stream H(3) comprising dissolved lithium hydroxide, into the upper part of the reactive distillation column K(3);
   (c.3.2) subjecting G(3) and H(3) in (K3) to reactive distillation conditions, obtaining a top stream W(3) comprising methanol and water; and obtaining a bottoms stream P(3) comprising lithium methoxide and methanol;
(d.1) feeding W(1), W(2) and W(3) into the lower part of a rectification column D, and feeding a stream M comprising methanol into the rectification column D;
(d.2) subjecting W(1), W(2), W(3) and M in D to distillation conditions, obtaining the stream G according to (a) as a top stream.

In particular as far as the steps (c.3) including (c.3.1) and (c.3.2), step (d.1) as far as W(3) is concerned and step (d.2) as far as W(3) is concerned, the skilled person, based on his general knowledge, will be in the position to derive from said details above in a straight-forward manner also any detail for operating the respective additional compressors, such as C(3) analogously to C(1) and C(2), the addition reactive distillation columns K(3) including, for example the additional droplet separator device, preferably the demister, and the preferred additional stream M(3) analogously to M(1) and M(2), and the like. At least two of the streams W(1), W(2) and W(3) may be suitably combined prior to being fed into D.

According to the present invention, it is also conceivable that from at least one of the streams P(i), A(i)OMe is at least partially separated from methanol, more preferably obtaining solid, more preferably crystalline A(i)OMe. Thus, solid, preferably crystalline sodium methoxide and solid, preferably crystalline potassium methoxide, can be obtained. According to the above-described process wherein 2 mixtures P(i) are prepared, it would also be preferred that, in addition, solid, preferably crystalline lithium methoxide can be obtained.

The present invention further relates to a chemical production unit for carrying out the process according to the present invention, comprising
a rectification column D comprising
   inlet means for feeding the stream M into D, preferably in the upper part of D;
   in its lower part, inlet means for feeding the streams W(i) or one or more combined stream thereof into D;
   outlet means for removing the streams T(2) and G or a combined stream thereof from the top of D;
   at least one condenser, preferably a condenser V(4) and optionally a further condenser V(5) arranged downstream of V(4), having inlet means for receiving the stream T(2) and having outlet means for removing the condensed stream T(3) and for removing a waste gas stream;
   inlet means for feeding the stream T(3) to the top of D;
   a bottom reboiler;
a stream dividing device S for dividing the stream G into n streams G(i);
means for passing the stream G to said stream dividing device S;
n reactive distillation columns K(i), n≥2 and i=1 . . . n; each reactive distillation column K(i) comprising
   in its upper part, preferably in its top, inlet means for feeding a stream H(i) into K(i);
   in its lower part, inlet means for feeding a stream G(i) into K(i);
   outlet means for removing a stream W(i) from the top of K(i);
   a bottom reboiler;
   outlet means for removing a bottoms stream from K(i);
   a stream dividing means for separating a stream P(i) from the bottoms stream removed from K(i);
means for passing the streams G(i) to the reactive distillation columns K(i);
means for passing the streams W(i) to the rectification column D;
one or more compressors C(i) for compressing either the stream G and/or the streams G(i) and/or the streams W(i).

Preferably, the top of at least one, more preferably of each reactive distillation column K(i) is equipped with a droplet separating device D(i), more preferably a demister, said demister more preferably comprising an inlet means for feeding a stream M(i) comprising methanol into said demister. Preferably each of the reactive distillations columns K(i) comprises, independently from one another, from 5 to 50, more preferably from 10 to 40, more preferably from 15 to 30 theoretical stages. It is preferred that the means for passing the streams G(i) to the reactive distillation columns K(i) are located, independently from one another, at a position between the bottoms and the fifth theoretical stage, more preferably between the bottoms and the third theoretical stage, more preferably between the bottoms and the second theoretical stage of K(i). Preferably, the means for passing the streams H(i) into the reactive distillation columns K(i) are located at the top of K(i), preferably at the uppermost theoretical stage. Preferably at least one, more preferably each reactive distillation columns K(i) does not comprise means for being operated at a reflux ratio of greater than 0:1. Preferably each reactive distillation column K(i) is equipped with trays.

It is preferred that the unit of the present invention comprises n compressors C(i) arranged upstream of K(i) for compressing the streams G(i). Alternatively, it is preferred that the unit of the present invention comprises n compressors C(i) arranged downstream of K(i) and upstream of D for compressing the streams W(i).

As to the rectification column D, it is preferred that said column has from 20 to 100, more preferably from 30 to 80, more preferably from 40 to 60 theoretical stages. Preferably, the inlet means of D for feeding the streams W(i) or one or more combined stream thereof into D is/are located at a position between the bottoms and the 15^{th} theoretical stage, more preferably between the bottoms and the 10^{th} theoretical stage, more preferably between the bottoms and the 8^{th} theoretical of D. It is preferred that the inlet means for feeding the stream M into D are located at least 4 theoretical stages from the top of D, more preferably between the 4^{th} and the 20^{th} theoretical stage from the top of D, more preferably between the 6^{th} and the 15^{th} theoretical stage from the top of D.

It is preferred that the unit of the present invention further comprises at least one condensate drum for a liquid stream removed from V(4) and optionally from V(5) and further comprising means for passing at least part of the liquid contained in said drum as the stream T(3) to the top of D. It is further preferred that the unit of the present invention also comprises means for realizing recompression of the top vapor obtained from D, said means more preferably comprising a compressor C(3) for compressing a stream T(1) removed from the top of D, means for passing T(1) from the top of D to C(3), a reboiler V(6) for condensing said compressed stream, means for passing said compressed stream from (C3) to V(6), and means for feeding the obtained liquid stream to the top of D. Preferably, the reboiler V(6) is a reboiler of D, more preferably an intermediate reboiler of D. It is further preferred that the rectification column D is equipped with trays and/or packings, wherein, insofar as the reboiler V(6) is a reboiler of D, D is more preferably equipped with packings arranged above the intermediate reboiler of D and with trays arranged below the intermediate reboiler of D.

In the context of the present invention, it is also conceivable that suitable reactive distillation columns K(i) are essentially bubble cap tray, valve tray and sieve tray columns. Specifically in the case of valve trays and sieve trays, the trays should be configured so that the raining-through of the liquid is minimized. A person skilled in the art will be familiar with the constructional measures required for this. Particularly tightly closing valve types are selected and thus, in particular, the vapor velocity into the tray openings is increased to double the value which is customarily set. This is achieved by a reduction in the number of valves. In the case of sieve trays, it is particularly useful to reduce the diameter of the openings in the tray and to maintain or even increase the number of openings. It is also conceivable that the columns are provided with random packing elements or ordered packing, with ordered packing being preferred over random packing elements with a view to uniform distribution of the liquid. The average ratio of liquid flow to vapor flow must not be exceeded by more than 15%, preferably not by more than 3%, in all sub-regions of the column cross section which correspond to more than 2% of the total column cross section. This low amount of liquid to be maintained makes it possible for the capillary effect on the wire meshes to prevent local peak values of the liquid trickle density.

According to the present invention, it is preferred that the unit further comprises at least one condensate drum for the condensed stream removed from V(6), the unit more preferably further comprising means for passing at least part of a gas phase in said drum to V(4) and means for passing at least part of a liquid phase in said drum to a condensate drum as defined in the foregoing.

Further, it may be preferred that the unit further comprises means for separating an alkali metal methoxide A(i)OMe from at least one of the streams P(i).

Preferably, the number of reactive distillation columns K(i), n, is in the range of from 2 to 10, more preferably in the range of from 2 to 5, more preferably 2 or 3, more preferably 2.

The present invention further relates to a use of a chemical production unit according to the present invention or of a process according to the present invention for simultaneously producing n mixtures P(i) comprising alkali metal methoxide and methanol, n being an integer with n≥2 and i=1 . . . n, wherein either at least 2 of the mixtures P(i) comprise different alkali metal methoxides A(i)OMe, and/or at least 2 of the mixtures P(i) comprise the same alkali metal alkoxide A(i)OMe at different concentrations.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3 and 4”.

Further, it is explicitly noted that the following set of embodiments is not the set of claims determining the extent of protection, but represents a suitably structured part of the description directed to general and preferred aspects of the present invention.
1. An integrated process for simultaneously preparing n mixtures P(i) comprising alkali metal methoxide and methanol, comprising
   providing n reactive distillation columns K(i);
   providing n aqueous liquid streams H(i), a given stream H(i) comprising a dissolved alkali metal hydroxide A(i)OH, wherein n is an integer with n≥2 and i=1 . . . n; and
   providing a rectification column D;
   the process further comprising
   (a) providing a stream G comprising methanol;
   (b) dividing the stream G into n streams G(i), each stream G(i) having the same composition as G;
   (c) preparing the one or more alkali metal methoxides comprising
      feeding each stream G(i) into the lower part of a respective reactive distillation column K(i), and feeding the aqueous liquid stream H(i) comprising the dissolved alkali metal hydroxide A(i)OH into the upper part of said reactive distillation column K(i); and
      subjecting G(i) and H(i) in each K(i) to reactive distillation conditions, obtaining n top streams W(i) comprising methanol and water; and obtaining n bottoms streams P(i) comprising alkali metal methoxide A(i)OMe and methanol;
   (d) feeding each stream W(i) into the lower part of the rectification column D, and feeding a stream M comprising methanol into the rectification column D; and subjecting the n streams W(i) and M in D to distillation conditions, obtaining the stream G according to (a) as a top stream.
2. The process of embodiment 1, wherein n is in the range of from 2 to 10, preferably in the range of from 2 to 5, more preferably 2 or 3, more preferably 2;
3. The process of embodiment 1 or 2, wherein each alkali metal hydroxide A(i)OH is preferably selected from the group consisting of lithium hydroxide, sodium hydroxide and potassium hydroxide, more preferably from the group consisting of sodium hydroxide and potassium hydroxide.
4. The process of any one of embodiments 1 to 3, wherein at least 2 of the alkali metal hydroxides A(i)OH are different from each other, wherein preferably, in particular if n is 2 or 3, all alkali metal hydroxides A(i)OH are different from each other.
5. The process of any one of embodiments 1 to 4, wherein a given liquid stream H(i) comprises an alkali metal hydroxide A(i)OH dissolved in water, in methanol, or in a mixture comprising water and methanol, preferably in water.
6. The process of any one of embodiments 1 to 5, being an integrated process for simultaneously preparing at least 2, preferably 2 mixtures P(i), wherein the mixture P(1) comprises A(1)OMe, preferably sodium methoxide, and methanol and the mixture P(2) comprises A(2)OMe, preferably potassium methoxide, and methanol, the process comprising
   (a) providing a stream G comprising methanol;
   (b) dividing the stream G into at least two streams G(1) and G(2), G(1) and G(2) having the same composition as G;
   (c.1) preparing A(1)OMe, comprising
      (c.1.1) feeding the stream G(1) into the lower part of a reactive distillation column K(1), and feeding a liquid stream H(1), preferably an aqueous liquid stream H(1), H(1) comprising dissolved A(1)OH, preferably sodium hydroxide, into the upper part of the reactive distillation column K(1);
      (c.1.2) subjecting G(1) and H(1) in K(1) to reactive distillation conditions, obtaining a top stream W(1) comprising methanol and water; and obtaining a bottoms stream P(1) comprising A(1)OMe and methanol;
   (c.2) preparing A(2)OMe, comprising
      (c.2.1) feeding the stream G(2) into the lower part of a reactive distillation column K(2), K(2) being arranged in parallel with K(1), and feeding a liquid stream H(2), preferably an aqueous liquid stream H(2), H(2) comprising dissolved A(2)OH, preferably potassium hydroxide, into the upper part of the reactive distillation column K(2);
      (c.2.2) subjecting G(2) and H(2) in K(2) to reactive distillation conditions, obtaining a top stream W(2) comprising methanol and water; and obtaining a bottoms stream P(2) comprising A(2)OMe and methanol;
   (d.1) feeding W(1) and W(2) into the lower part of a rectification column D, and feeding a stream M comprising methanol into the rectification column D;
   (d.2) subjecting W(1), W(2) and M in D to distillation conditions, obtaining the stream G according to (a) as a top stream.
7. The process of embodiment 6, wherein W(1) and W(2) are fed as gas streams into the rectification column D, preferably at a position between the bottoms and the 15^{th} theoretical stage, preferably between the bottoms and the 10^{th} theoretical stage, more preferably between the bottoms and the 8^{th} theoretical of the rectification column D.
8. The process of any one of embodiments 1 to 7, wherein the rectification column D has from 20 to 100, preferably from 30 to 80, more preferably from 40 to 60 theoretical stages.
9. The process of any one of embodiments 1 to 8, wherein the rectification column D is operated at a pressure at the top in the range of from 0.5 to 10 bar(abs), preferably in the range of from 0.75 to 6 bar(abs), more preferably in the range of from 1 to 5 bar(abs).
10. The process of any one of embodiments 1 to 9, wherein from 99 to 100 weight-%, preferably from 99.5 to 100 weight-%, more preferably from 99.9 to 100 weight-% of the stream M consist of methanol and optionally water, wherein the amount of water comprised in the stream M is at most 2000 weight-ppm, more preferably at most 1500 weight-ppm, more preferably at most 1000 weight-ppm.
11. The process of any one of embodiments 1 to 10, wherein the stream M is fed to the upper part of D, preferably at least 4 theoretical stages from the top of D, more preferably between the 4^{th} and the 20^{th} theoretical stage from the top of D, more preferably between the 6^{th} and the 15^{th} theoretical stage from the top of D, preferably at a temperature of M in the range of from ambient temperature to the boiling point of methanol at the column pressure of D, more preferably at ambient temperature.
12. The process of any one of embodiments 1 to 11, wherein the rectification column D is operated at a reflux ratio of at least 0.5:1, preferably in the range of from 0.55:1 to 1.4:1, more preferably in the range of from 0.6:1 to 1.4:1.
13. The process of embodiment 12, wherein the rectification column D is operated without top vapor recompression.
14. The process of embodiment 13, wherein realizing the reflux ratio comprises removing, in addition to G, a top stream T(2) from the rectification column, passing said stream T(2) through a condenser V(4), obtaining a liquid stream T(3) and a waste gas stream T(2w), and feeding the liquid stream T(3) into the top of the rectification column D.
15. The process of embodiment 13, wherein realizing the reflux ratio comprises removing, in addition to G, a top stream T(2) from the rectification column, passing said stream T(2) through a condenser V(4), obtaining a liquid stream T(2l) and a gas stream T(2g); passing the gas stream T(2g) through a condenser V(5), obtaining a liquid stream T(2gl) and a waste gas stream T(2w); and combining the liquid streams T(2l) and (T2gl), obtaining a combined liquid stream which is fed as the stream T(3) into the top of the rectification column D.
16. The process of embodiment 14 or 15, wherein the waste gas stream T(2w) essentially consists of oxygen, nitrogen, carbon dioxide and methanol, wherein the amount of methanol in T(2w) is preferably in the range of from 2 to 80 weight-%, preferably in the range of from 10 to 30 weight-% based on the total weight of T(2w).
17. The process of embodiment 12, wherein the rectification column is operated with top vapor recompression.
18. The process of embodiment 17, wherein realizing the reflux ratio comprises
   (i) removing, in addition to G, a top stream T(2) from the rectification column D, passing said stream T(2) through a condenser V(4), obtaining a liquid stream and a waste gas stream T(2w);
   (ii) removing, in addition to G and T(2), a further top stream T(1) from the rectification column D, passing said stream T(2) through a compressor C(3), passing the thus compressed stream through a reboiler V(6), obtaining a liquid stream, wherein the reboiler V(6) is preferably a reboiler of the rectification column D;
   (iii) feeding the liquid streams obtained according to (i) and (ii) into the top of the rectification column D.
19. The process of embodiment 17, wherein realizing the reflux ratio comprises
   (i) removing, in addition to G, a top stream T(2) from the rectification column D, passing said stream T(2) through a condenser V(4), obtaining a liquid stream T(2l) and a gas stream T(2g); passing the gas stream T(2g) through a condenser V(5), obtaining a liquid stream T(2gl) and a waste gas stream T(2w); and combining the liquid streams T(2l) and (T2gl), obtaining a combined liquid stream T(2cl);
   (ii) removing, in addition to G and T(2), a further top stream T(1) from the rectification column D, passing said stream T(2) through a compressor C(3), passing the thus compressed stream through a reboiler V(6), obtaining a liquid stream, wherein the reboiler V(6) is preferably a reboiler of the rectification column D;
   (iii) feeding the combined liquid stream obtained according to (i) and the liquid stream obtained according to (ii) into the top of the rectification column D.
20. The process of embodiment 18 or 19, wherein the waste gas stream T(2w) essentially consists of oxygen, nitrogen, carbon dioxide and methanol, wherein the amount of methanol in T(2w) is preferably in the range of from 2 to 80 weight-%, preferably in the range of from 10 to 30 weight-% based on the total weight of T(2w).
21. The process of any one of embodiments 18 to 20, wherein (ii) further comprises feeding the liquid stream obtained from the reboiler V(6) to a condensate drum, wherein from said condensate drum, a gas stream T(1g) and a liquid stream T(11) are removed, said gas stream T(1g) being fed into the condenser V(4) and said liquid stream T(11) the liquid stream obtained according to (ii), wherein prior to being fed into the top of the rectification column D according to (iii), the liquid stream is preferably depressurized.
22. The process of any one of embodiments 18 to 21, wherein (iii) comprises combining the liquid streams obtained according to (i) and (ii) to obtain a liquid stream T(3) and feeding the stream T(3) into the top of the rectification column D.
23. The process of any one of embodiments 18 to 22, wherein according to (ii), the reboiler V(6) is an intermediate reboiler of the rectification column D or the bottom reboiler of the rectification column D, preferably an intermediate reboiler of the rectification column D.
24. The process of any one of embodiments 1 to 23, wherein the stream G provided according to (a) by obtaining from distillation according to (d.2) comprises methanol and water, wherein preferably from 99.95 to 100 weight-% of G consist of methanol and water, and wherein the water content of G is at most 200 weight-ppm, preferably at most 150 weight-ppm, more preferably at most 100 weight-ppm, wherein more preferably said water content is in the range of from 5 to 100 weight-ppm, more preferably in the range of from 10 to 100 weight-ppm, more preferably in the range of from 15 to 100 weight-ppm.
25. The process of any one of embodiments 1 to 24, wherein according to (b), the stream G is divided into the two streams G(1) and G(2), wherein the stream G has a mass flow rate f(G), the stream G(1) has a mass flow rate f(G(1)) and the stream G(2) has a mass flow rate f(G(2)), wherein f(G)=f(G(1))+f(G(2)).
26. The process of any one of embodiments 1 to 25, wherein dividing according to (b) comprises passing the stream G into a stream dividing device S, said device preferably comprising a pipe junction.
27. The process of embodiment 25 or 26, wherein the ratios f(G(1))/f(G) and f(G(2))/f(G) are adjusted depending on the desired amount of A(1)OMe to be obtained according to (c.1.2) relative to the desired amount of A(2)OMe to be obtained according to (c.2.2).
28. The process of any one of embodiments 1 to 27, wherein prior to dividing according to (b), the stream G is passed through a compressor C, thereby realizing a pressure increase of G in the range of from 0.1 to 0.8 bar(abs), preferably in the range of from 0.15 to 0.6 bar(abs), more preferably in the range of from 0.2 to 0.4 bar(abs).
29. The process of embodiment 28, wherein dividing according to (b) comprises passing the compressed stream G into a stream dividing device S, said device preferably comprising a pipe junction and at least one control device allowing for adjusting the ratio f(G(1))/f(G(2)) as defined in embodiment 25, said at least one control device being located downstream of said pipe junction, wherein at least one of these control devices is located either in the stream G(1) or in the stream G(2) or in both streams G(1) and G(2), wherein said at least one control device preferably comprises a control valve.
30. The process of any one of embodiments 1 to 29, preferably of any one of embodiments 1 to 27, wherein prior to be fed into the reactive distillation column K(1), the stream G(1) is passed through a compressor C(1), thereby realizing a pressure increase of G(1) in the range of from 0.1 to 0.8 bar(abs), preferably in the range of from 0.15 to 0.6 bar(abs), more preferably in the range of from 0.2 to 0.4 bar(abs).
31. The process of any one of embodiments 1 to 30, preferably of any one of embodiments 1 to 27, wherein prior to be fed into the reactive distillation column K(2), the stream G(2) is passed through a compressor C(2), thereby realizing a pressure increase of G(2) in the range of from 0.1 to 0.8 bar(abs), preferably in the range of from 0.15 to 0.6 bar(abs), more preferably in the range of from 0.2 to 0.4 bar(abs).
32. The process of any one of embodiments 1 to 31, wherein from 99 to 100 weight-%, more preferably from 99.5 to 100 weight-%, more preferably from 99.9 to 100 weight-% of the stream H(1) consist of water and A(1)OH, wherein preferably from 37.5 to 58 weight-%, more preferably from 40 to 55 weight-%, more preferably from 42.5 to 52 weight-% of the stream H(1) consist of A(1)OH.
33. The process of any one of embodiments 1 to 32, wherein the stream H(1) is fed into the reactive distillation column K(1) at a temperature of H(1) in the range of from ambient temperature to its boiling temperature, preferably in the range of from 50 to 80° C.
34. The process of any one of embodiments 1 to 33, wherein the stream H(1) is fed into the top of the reactive distillation column K(1), preferably to the first theoretical stage from the top.
35. The process of any one of embodiments 1 to 34, wherein the reactive distillation column K(1) has from 5 to 50, preferably from 10 to 40, more preferably from 15 to 30 theoretical stages, wherein the stream G(1) is fed into the reactive distillation column K(1) at a position preferably between the bottoms and the fifth theoretical stage, more preferably between the bottoms and the third theoretical stage, more preferably between the bottoms and the second theoretical stage of the reactive distillation column K(1).
36. The process of any one of embodiments 1 to 35, wherein the reactive distillation column K(1) is operated at a pressure at the top in the range of from 0.5 to 10 bar(abs), preferably in the range of from 1 to 6 bar(abs), more preferably in the range of from 1 to 5 bar(abs).
37. The process of any one of embodiments 1 to 36, wherein the reactive distillation column K(1) is operated at a reflux ratio of 0:1.
38. The process of any one of embodiments 1 to 37, wherein from 99 to 100 weight-%, preferably from 99.5 to 100 weight-%, more preferably from 99.9 to 100 weight-% of the stream W(1) consist of methanol and water, wherein preferably from 1 to 10 weight-%, more preferably from 2 to 8 weight-%, more preferably from 5 to 6 weight-% of the stream W(1) consist of water.
39. The process of any one of embodiments 1 to 38, wherein from 99 to 100 weight-%, preferably from 99.5 to 100 weight-%, more preferably from 99.9 to 100 weight-% of the stream P(1) consist of A(1)OMe and methanol, wherein preferably from 10 to 50 weight %, more preferably from 20 to 40 weight-%, more preferably from 25 to 35 weight-% of the stream P(1) consist of A(1)OMe, and wherein preferably at most 5000 weight-ppm, more preferably at most 2000 weight-ppm, more preferably at most 1000 weight-ppm of the stream P(1) consist of water.
40. The process of any one of embodiments 1 to 39, wherein the reactive distillation column K(1) is operated at a reboiler duty allowing to achieve a specific concentration of A(1)OMe in the stream P(1), preferably the concentration as defined in embodiment 39.
41. The process of any one of embodiments 1 to 40, wherein the top of the reactive distillation column K(1) is equipped with a droplet separating device D(1), preferably a demister, the process comprising separating droplets comprising A(1)OH from the vapor stream in the top of K(1).
42. The process of embodiment 41, comprising at least temporarily spraying the demister, preferably with a stream M(1) comprising methanol, said stream preferably being branched from a condensed top stream removed from the rectification column D or being a fresh methanol stream.
43. The process of any one of embodiments 1 to 42, wherein from 99 to 100 weight-%, more preferably from 99.5 to 100 weight-%, more preferably from 99.9 to 100 weight-% of the stream H(2) consist of water and A(2)OH, wherein preferably from 30 to 55 weight-%, more preferably from 40 to 52.5 weight-%, more preferably from 45 to 50 weight-% of the stream H(2) consist of A(2)OH.
44. The process of any one of embodiments 1 to 43, wherein the stream H(2) is fed into the reactive distillation column K(2) at a temperature of H(2) in the range of from ambient temperature to its boiling temperature, preferably in the range of from 50 to 80° C.
45. The process of any one of embodiments 1 to 44, wherein the stream H(2) is fed into the top of the reactive distillation column K(2), preferably to the first theoretical stage from the top.
46. The process of any one of embodiments 1 to 45, wherein the reactive distillation column K(2) has from 5 to 50, preferably from 10 to 40, more preferably from 15 to 30 theoretical stages, wherein the stream G(2) is fed into the reactive distillation column K(2) at a position preferably between the bottoms and the fifth theoretical stage, more preferably between the bottoms and the third theoretical stage, more preferably between the bottoms and the second theoretical stage of the reactive distillation column K(2).
47. The process of any one of embodiments 1 to 46, wherein the reactive distillation column K(2) is operated at a pressure at the top in the range of from 0.5 to 10 bar(abs), preferably in the range of from 1 to 6 bar(abs), more preferably in the range of from 1 to 5 bar(abs)
48. The process of any one of embodiments 1 to 47, wherein the reactive distillation column K(2) is operated at a reflux ratio of 0:1.
49. The process of any one of embodiments 1 to 48, wherein from 99 to 100 weight-%, preferably from 99.5 to 100 weight-%, more preferably from 99.9 to 100 weight-% of the stream W(2) consist of methanol and water, wherein preferably from 1 to 15 weight-%, more preferably from 2 to 12 weight-%, more preferably from 6 to 10 weight-% of the stream W(2) consist of water.
50. The process of any one of embodiments 1 to 49, wherein from 99 to 100 weight-%, preferably from 99.5 to 100 weight-%, more preferably from 99.9 to 100 weight-% of the stream P(2) consist of A(2)OMe and methanol, wherein preferably from 10 to 50 weight %, more preferably from 20 to 40 weight-%, more preferably from 25 to 35 weight-% of the stream P(2) consist of A(2)OMe, and wherein preferably at most 5000 weight-ppm, more preferably at most 2000 weight-ppm, more preferably at most 1000 weight-ppm of the stream P(2) consist of water.
51. The process of any one of embodiments 1 to 50, wherein the reactive distillation column K(2) is operated at a reboiler duty allowing to achieve a specific concentration of A(2)OMe in the stream P(2), preferably the concentration as defined in embodiment 50.
52. The process of any one of embodiments 1 to 51, wherein the top of the reactive distillation column K(2) is equipped with a droplet separating device D(2), preferably a demister, the process comprising separating droplets comprising A(2)OH from the vapor stream in the top of K(2).
53. The process of embodiment 52, comprising at least temporarily spraying the demister, preferably with a stream M(2) comprising methanol, said stream preferably being branched from a condensed top stream removed from the rectification column D or being a fresh methanol stream.
54. The process of any one of embodiments 1 to 53, wherein prior to being fed into the rectification column D, the stream W(1) is passed through a compressor C(1), thereby realizing a pressure increase of W(1) in the range of from 0.1 to 0.8 bar(abs), preferably in the range of from 0.15 to 0.6 bar(abs), more preferably in the range of from 0.2 to 0.4 bar(abs).
55. The process of any one of embodiments 1 to 54, wherein prior to being fed into the rectification column D, the stream W(2) is passed through a compressor C(2), thereby realizing a pressure increase of W(2) in the range of from 0.1 to 0.8 bar(abs), preferably in the range of from 0.15 to 0.6 bar(abs), more preferably in the range of from 0.2 to 0.4 bar(abs).
56. The process of embodiment 54 or 55, wherein prior to dividing according to (b), the stream G is not passed through a compressor C, preferably not passed through a compressor C thereby realizing a pressure increase of G in the range of from 0.1 to 0.8 bar(abs), preferably in the range of from 0.15 to 0.6 bar(abs), more preferably in the range of from 0.2 to 0.4 bar(abs).
57. The process of any one of embodiments 54 to 56, wherein prior to be fed into the reactive distillation column K(1), the stream G(1) is not passed through a compressor C(1), preferably not passed through a compressor C(1) thereby realizing a pressure increase of G(1) in the range of from 0.1 to 0.8 bar(abs), preferably in the range of from 0.15 to 0.6 bar(abs), more preferably in the range of from 0.2 to 0.4 bar(abs).
58. The process of any one of embodiments 54 to 57, wherein prior to be fed into the reactive distillation column K(2), the stream G(2) is not passed through a compressor C(2), preferably not passed through a compressor C(2) thereby realizing a pressure increase of G(2) in the range of from 0.1 to 0.8 bar(abs), preferably in the range of from 0.15 to 0.6 bar(abs), more preferably in the range of from 0.2 to 0.4 bar(abs).
59. The process of any one of embodiments 1 to 58, being an integrated process for simultaneously preparing 2 mixtures P(i), wherein the mixture P(1) comprises sodium methoxide as A(1)OMe and methanol and the mixture P(2) comprising potassium methoxide as A(2)OMe and methanol.
60. The process of any one of embodiments 1 to 58, being an integrated process for simultaneously preparing three mixtures P(1), P(2) and P(3), wherein the mixture P(1) comprises sodium methoxide as A(1)OMe and methanol, the mixture P(2) comprises potassium methoxide as A(2)OMe and methanol, and the mixture P(3) comprises lithium methoxide as A(3)OMe and methanol, the process comprising
   (a) providing a stream G comprising methanol;
   (b) dividing the stream G into three streams G(1), G(2) and G(3), G(1), G(2) and G(3) having the same composition as G;
   (c.1) preparing sodium methoxide comprising
      (c.1.1) feeding the stream G(1) into the lower part of a reactive distillation column K(1), and feeding a liquid stream H(1), preferably an aqueous liquid stream H(1), H(1) comprising dissolved sodium hydroxide, into the upper part of the reactive distillation column K(1);
      (c.1.2) subjecting G(1) and H(1) in K(1) to reactive distillation conditions, obtaining a top stream W(1) comprising methanol and water; and obtaining a bottoms stream P(1) comprising sodium methoxide and methanol;
   (c.2) preparing potassium methoxide comprising
      (c.2.1) feeding the stream G(2) into the lower part of a reactive distillation column K(2), K(2) being arranged in parallel with K(1), and feeding a liquid stream H(2), preferably an aqueous liquid stream H(2), H(2) comprising dissolved potassium hydroxide, into the upper part of the reactive distillation column K(2);
      (c.2.2) subjecting G(2) and H(2) in K(2) to reactive distillation conditions, obtaining a top stream W(2) comprising methanol and water; and obtaining a bottoms stream P(2) comprising potassium methoxide and methanol;
   (c.3) preparing lithium methoxide comprising
      (c.3.1) feeding the stream G(3) into the lower part of a reactive distillation column K(3), K(3) being arranged in parallel with K(1) and K(2), and feeding a liquid stream H(3), preferably an aqueous liquid stream H(3), H(3) comprising dissolved lithium hydroxide, into the upper part of the reactive distillation column K(3);
      (c.3.2) subjecting G(3) and H(3) in (K3) to reactive distillation conditions, obtaining a top stream W(3) comprising methanol and water; and obtaining a bottoms stream P(3) comprising lithium methoxide and methanol;
   (d.1) feeding W(1), W(2) and W(3) into the lower part of a rectification column D, and feeding a stream M comprising methanol into the upper part of the rectification column D;
   (d.2) subjecting W(1), W(2), W(3) and M in D to distillation conditions, obtaining the stream G according to (a) as a top stream.
61. The process of any one of embodiments 1 to 60, wherein from at least one of the streams P(i), A(i)OMe is at least partially separated from methanol, preferably obtaining solid, more preferably crystalline A(i)OMe.
62. A chemical production unit for carrying out the process according to any one of embodiments 1 to 61, comprising
   a rectification column D comprising
      inlet means for feeding the stream M into D;
      in its lower part, inlet means for feeding the streams W(i) or one or more combined stream thereof into D;
      outlet means for removing the streams T(2) and G or a combined stream thereof from the top of D;
      at least one condenser, preferably a condenser V(4) and optionally a further condenser V(5) arranged downstream of V(4), having inlet means for receiving the stream T(2) and having outlet means for removing the condensed stream T(3) and for removing a waste gas stream;
      inlet means for feeding the stream T(3) to the top of D;
      a bottom reboiler;
   a stream dividing device S for dividing the stream G into n streams G(i);
   means for passing the stream G to said stream dividing device S;
   n reactive distillation columns K(i), n≥2 and i=1 . . . n; said reactive distillation columns K(i) being arranged in parallel, each reactive distillation column K(i) comprising
      in its upper part, preferably in its top, inlet means for feeding a stream H(i) into K(i);
      in its lower part, inlet means for feeding a stream G(i) into K(i);
      outlet means for removing a stream W(i) from the top of K(i);
      a bottom reboiler;
      outlet means for removing a bottoms stream from K(i);
      a stream dividing means for separating a stream P(i) from the bottoms stream removed from K(i);
   means for passing the streams G(i) to the reactive distillation columns K(i);
   means for passing the streams W(i) to the rectification column D;
   one or more compressors C(i) for compressing either the stream G and/or the streams G(i) and/or the streams W(i).
63. The unit of embodiment 62, wherein the top of at least one, preferably of each reactive distillation column K(i) is equipped with a droplet separating device D(i), preferably a demister, said demister preferably comprising an inlet means for feeding a stream M(i) comprising methanol into said demister.
64. The unit of embodiment 62 or 63, wherein each of the reactive distillations columns K(i) comprises, independently from one another, from 5 to 50, preferably from 10 to 40, more preferably from 15 to 30 theoretical stages.
65. The unit of any one of embodiments 62 to 64, wherein the means for passing the streams G(i) to the reactive distillation columns K(i) are located, independently from one another, at a position between the bottoms and the fifth theoretical stage, more preferably between the bottoms and the third theoretical stage, more preferably between the bottoms and the second theoretical stage of K(i).
66. The unit of any one of embodiments 62 to 65, wherein the means for passing the streams H(i) into the reactive distillation columns K(i) are located at the top of K(i), preferably at the uppermost theoretical stage.
67. The unit of any one of embodiments 62 to 66, wherein at least one, preferably each reactive distillation columns K(i) does not comprise means for being operated at a reflux ratio of greater than 0:1.
68. The unit of any one of embodiments 62 to 67, wherein each reactive distillation column K(i) is equipped with trays.
69. The unit of any one of embodiments 62 to 68, comprising n compressors C(i) arranged upstream of K(i) for compressing the streams G(i).
70. The unit of any one of embodiments 62 to 68, comprising n compressors C(i) arranged downstream of K(i) and upstream of D for compressing the streams W(i).
71. The unit of any one of embodiments 62 to 70, wherein the rectification column D has from 20 to 100, preferably from 30 to 80, more preferably from 40 to 60 theoretical stages.
72. The unit of any one of embodiments 62 to 71, wherein the inlet means for feeding the streams W(i) or one or more combined stream thereof into D is/are located at a position between the bottoms and the 15^{th} theoretical stage, preferably between the bottoms and the 10^{th} theoretical stage, more preferably between the bottoms and the 8^{th} theoretical of D.
73. The unit of any one of embodiments 62 to 72, wherein the inlet means for feeding the stream M into D are located in the upper part of D, preferably at least 4 theoretical stages from the top of D, more preferably between the 4^{th} and the 20^{th} theoretical stage from the top of D, more preferably between the 6^{th} and the 15^{th} theoretical stage from the top of D.
74. The unit of any one of embodiments 62 to 73, further comprising at least one condensate drum for a liquid stream removed from V(4) and optionally from V(5) and further comprising means for passing at least part of the liquid contained in said drum as the stream T(3) to the top of D.
75. The unit of any one of embodiments 62 to 74, further comprising means for realizing recompression of the top vapor obtained from D, said means preferably comprising a compressor C(3) for compressing a stream T(1) removed from the top of D, means for passing T(1) from the top of D to C(3), a reboiler V(6) for condensing said compressed stream, means for passing said compressed stream from (C3) to V(6), and means for feeding the obtained liquid stream to the top of D.
76. The unit of embodiment 75, wherein the reboiler V(6) is a reboiler of D, preferably an intermediate reboiler of D.
77. The unit of any one of embodiments 62 to 76, wherein the rectification column is equipped with trays and/or packings, wherein, insofar as embodiment 77 is dependent on embodiment 76, D is equipped with packings arranged above the intermediate reboiler of D and with trays arranged below the intermediate reboiler of D.
78. The unit of embodiment 76 or 77, further comprising at least one condensate drum for the condensed stream removed from V(6), the unit preferably further comprising means for passing at least part of a gas phase in said drum to V(4) and means for passing at least part of a liquid phase in said drum to a condensate drum according to embodiment 74.
79. The unit of any one of embodiments 62 to 78, further comprising means for separating an alkali metal methoxide A(i)OMe from at least one of the streams P(i).
80. The unit of any one of embodiments 62 to 79, wherein n is in the range of from 2 to 10, preferably in the range of from 2 to 5, more preferably 2 or 3, more preferably 2.
81. Use of a chemical production unit according to any one of embodiments 62 to 80 or of a process according to any one of embodiments 1 to 61 for simultaneously producing n mixtures P(i) comprising alkali metal methoxide and methanol, n being an integer with n≥2 and i=1 . . . n, wherein either at least 2 of the mixtures P(i) comprise different alkali metal methoxides A(i)OMe, and/or at least 2 of the mixtures P(i) comprise the same alkali metal alkoxide A(i)OMe at different concentrations.

The present invention is further illustrated by the following examples 1 and 2 and the FIGS. 1 to 6.

### EXAMPLES

### Example 1: Simultaneous Production of Sodium Methoxide and Potassium Methoxide without Top Vapor Recompression in Rectification Column D

FIG. 2 shows a process scheme for preparing a mixture P(1) comprising NaOMe and MeOH and a mixture P(2) comprising KOMe and MeOH. Regarding the operating conditions of the rectification column D and of the reactive distillation columns K(1) and K(2), reference is made to Table 1a below. Regarding the relative mass flow rates, reference is made to Table 1 b below.

TABLE 1a
Operating conditions of the columns D, K(1) and K(2)

| | | |
|---|---|---|
| Column D | Pressure at the top/bar(abs) | 2.1 |
| | Temperature at the top/° C. | 84 |
| | Pressure at the bottom/bar(abs) | 2.23 |
| | Temperature at the bottom/° C. | 124 |
| | Theoretical stages | 50 |
| | W(1), W(2) to theoretical stage from bottom | 8th |
| | M fed to theoretical stage from bottom | 42th |
| Column K(1) | Pressure at the top/bar(abs) | 2.15 |
| | Temperature at the top/° C. | 89 |
| | Pressure at the bottom/bar(abs) | 2.3 |
| | Temperature at the bottom/° C. | 117 |
| | Number of trays | 40 |
| Column K(2) | Pressure at the top/bar(abs) | 2.15 |
| | Temperature at the top/° C. | 89 |
| | Pressure at the bottom/bar(abs) | 2.3 |
| | Temperature at the bottom/° C. | 116 |
| | Number of trays | 40 |

TABLE 1b
Relationships between the mass flow rates f of the different streams

| Definition of | Specified: | H(1), H(2) |
|---|---|---|
| streams | M(1), M(2): | part of condensate from V(4) |
| | H(1): | NaOH 50 weight-% in water |
| | H(2): | KOH 48 weight-% in water |
| Ratios of | f(G(1))/f(H(1)) | 13.3 |
| mass flow rates | f(G(2))/f(H(2)) | 9.03 |
| of streams | f(M(1))/f(H(1)) | 0.55 |
| | f(M(2))/f(H(2)) | 0.40 |
| | f(T(3))/f(G) *) | 0.92 |
| | f(P(1))/f(H(1)) | 2.25 |
| | f(P(2))/f(H(2)) | 1.86 |
| | f(waste gas)/f(G) *) | <0.0015 |

*) f(G) = f(G(1)) + f(G(2))

P(1): 30 weight-% of sodium methoxide in methanol, <1000 ppm of water.
P(2): 32 weight-% of potassium methoxide in methanol, <1000 ppm of water.

In the following, it is indicated how the mass flow rate of the methanol contained in the stream M (methanol balance, fresh methanol stream), f_{MeOH}(M), is calculated. In this calculation, the water contents of P(1) and P(2), both being less than 1000 weight-ppm, are neglected. According to this calculation, f_{MeOH}(P(1)) is the mass flow rate of MeOH contained in the stream P(1), f_{MeOH}(P(2)) is the mass flow rate of MeOH contained in the stream P(2), f_{MeOH}(water) is the mass flow rate of MeOH contained in the water stream, and f_{MeOH}(waste gas) is the mass flow rate of MeOH contained in the waste gas stream:

f_{MeOH}(M)=f_{MeOH}(P(1))+f_{MeOH}(P(2))+f_{MeOH}(water)+f_{MeOH}(waste gas)

N_{MeOH}(P(1))=[(1−c_{NaoME})*f(P(1))]+[(M_{MeOH}/M_{NaOH}*c_{NaOH})*f(H(1))] 1.1

| Molecular mass M/concentration c | values | units |
|---|---|---|
| M_{MeOH} | 32 | kg/kmol |
| M_{NaOH} | 40 | kg/kmol |
| c_{NaOH} in H(1) | 0.5 | kg/kg |
| c_{NaOMe} in P(1) | 0.3 | kg/kg |

f_{MeOH}(P(2))=[(1−c_{KOMe})*f(P(2))]+[(M_{MeOH}/M_{KOH}*c_{KOH})*f(H(2))] 1.2

| Molecular mass M/concentration c | values | units |
|---|---|---|
| M_{MeOH} | 32 | kg/kmol |
| M_{KOH} | 56 | kg/kmol |
| c_{KOH} in H(2) | 0.48 | kg/kg |
| c_{KOMe} in P(2) | 0.32 | kg/kg |

f_{MeOH}(water)=0.001*f(water)(maximum value) 1.3

f_{MeOH}(waste gas)=0(neglected) 1.4

In the following, it is indicated how the mass flow rate of the water contained in the stream water (bottom stream of D, waste water stream), f(water), is calculated. In this calculation, the water contents of P(1) and P(2), both being less than 1000 weight-ppm, are neglected.

f(water)=f_{H2O}(H(1))+f_{H2O}(H(2))+f_{H2O}(M)−f_{H2O}(waste gas) 1.5
f_{H2O}(H(1)) is the mass flow rate of water contained in the stream H(1) and f_{H2O}(H(2)) is the mass flow rate of water contained in the stream H(2) and f_{H2O}(M) is the mass flow rate of water contained in the stream M:

| Molecular mass M/concentration c | values | units |
|---|---|---|
| M_{H2O} | 18 | kg/kmol |
| M_{NaOH} | 40 | kg/kmol |
| c_{NaOH} in H(1) | 0.5 | kg/kg |

f_{H2O}(H(1))=[(1−c_{NaOH})*f(H(1))]+[(M_{H2O}/M_{NaOH}*c_{NaOH})*f(H(1))] 1.5.1

| Molecular mass M/concentration c | values | units |
|---|---|---|
| M_{H2O} | 18 | kg/kmol |
| M_{KOH} | 56 | kg/kmol |
| c_{KOH} in H(2) | 0.48 | kg/kg |

f_{H2O}(H(2))=[(1−c_{KOH})*f(H(2))]+[(M_{H2O}/M_{KOH}*c_{KOH})*f(H(2))] 1.5.2

f_{H2O}(M)=0.001*f(M) 1.5.3

f_{H2O}(waste gas)=0(neglected) 1.5.4

### Example 2: Simultaneous Production of Sodium Methoxide and Potassium Methoxide with Top Vapor Recompression in Rectification Column D

The use of vapor recompression reduces the energy demand of the distillation in D considerably. It is possible to have a ratio of the heat streams to V(3) and V(6) of about 1:4. That means the energy demand decreases to 20%. But about 10% (depending on the pressure) of the energy which is transferred in V(6) is needed as power for the compressor C(3). All in all, there is a large energy saving by using vapor recompression.

FIG. 5 shows a process scheme for preparing a mixture P(1) comprising NaOMe and MeOH and a mixture P(2) comprising KOMe and MeOH. Regarding the operating conditions of the rectification column D and of the reactive distillation columns K(1) and K(2), reference is made to Table 2a below. Regarding the relative mass flow rates, reference is made to Table 2b below.

TABLE 2a
Operating conditions of the columns D, K(1) and K(2)

| | | |
|---|---|---|
| Column D | Pressure at the top/bar(abs) | 2.1 |
| | Temperature at the top/° C. | 84 |
| | Pressure at the bottom/bar(abs) | 2.23 |
| | Temperature at the bottom/° C. | 124 |
| | Theoretical stages | 50 |
| | W(1), W(2) to theoretical stage from bottom | 8th |
| | M fed to theoretical stage from bottom | 42th |
| | Pressure at outlet of C(3)/bar(abs) | 5 |
| Column K(1) | Pressure at the top/bar(abs) | 2.15 |
| | Temperature at the top/° C. | 89 |
| | Pressure at the bottom/bar(abs) | 2.3 |
| | Temperature at the bottom/° C. | 117 |
| | Number of trays | 40 |
| Column K(2) | Pressure at the top/bar(abs) | 2.15 |
| | Temperature at the top/° C. | 89 |
| | Pressure at the bottom/bar(abs) | 2.3 |
| | Temperature at the bottom/° C. | 116 |
| | Number of trays | 40 |

TABLE 2b
Relationships between the mass flow rates f of the different streams

| Definition of | Specified: | H(1), H(2) |
|---|---|---|
| streams | M(1), M(2): | part of condensate from V(4) |
| | H(1): | NaOH 50 weight-% in water |
| | H(2): | KOH 48 weight-% in water |
| Ratios of | f(G(1))/f(H(1)) | 13.3 |
| mass flow rates | f(G(2))/f(H(2)) | 9.03 |
| of streams | f(M(1))/f(H(1)) | 0.55 |
| | f(M(2))/f(H(2)) | 0.40 |
| | f(T(3))/f(G) *) | 1.0 |
| | f(P(1))/f(H(1)) | 2.25 |
| | f(P(2))/f(H(2)) | 1.86 |
| | f(waste gas)/f(G) *) | <0.0015 |
| | f(T(1))/f(T(2)) | 3.97 |

*) f(G) = f(G(1)) + f(G(2))

P(1): 30 weight-% of sodium methoxide in methanol, <1000 ppm of water.
P(2): 32 weight-% of potassium methoxide in methanol, <1000 ppm of water.

In the following, it is indicated how the mass flow rate of the methanol contained in the stream M (methanol balance, fresh methanol stream), f_{MeOH}(M), is calculated. In this calculation, the water contents of P(1) and P(2), both being less than 1000 weight-ppm, are neglected. According to this calculation, f_{MeOH}(P(1)) is the mass flow rate of MeOH contained in the stream P(1), f_{MeOH}(P(2)) is the mass flow rate of MeOH contained in the stream P(2), f_{MeOH}(water) is the mass flow rate of MeOH contained in the water stream, and f_{MeOH}(waste gas) is the mass flow rate of MeOH contained in the waste gas stream:

f_{MeOH}(M)=f_{MeOH}(P(1))+f_{MeOH}(P(2))+f_{MeOH}(water)+f_{MeOH}(waste gas)

N_{MeOH}(P(1))=[(1−c_{NaOME})*f(P(1))]+[(M_{MeOH}/M_{NaOH}*c_{NaOH})*f(H(1))] 2.1

| Molecular mass M/concentration c | values | units |
|---|---|---|
| M_{MeOH} | 32 | kg/kmol |
| M_{NaOH} | 40 | kg/kmol |
| c_{NaOH} in H(1) | 0.5 | kg/kg |
| c_{NaOMe} in P(1) | 0.3 | kg/kg |

N_{MeOH}(P(2))=[(1−c_{KOMe})*f(P(2))]+[(M_{MeOH}/M_{KOH}*c_{KOH})*f(H(2))] 2.2

| Molecular mass M/concentration c | values | units |
|---|---|---|
| M_{MeOH} | 32 | kg/kmol |
| M_{KOH} | 56 | kg/kmol |
| c_{KOH} in H(2) | 0.48 | kg/kg |
| c_{KOMe} in P(2) | 0.32 | kg/kg |

f_{MeOH}(Water)=0.001*f(water)(maximum value) 2.3

f_{MeOH}(waste gas)=0(neglected) 2.4

In the following, it is indicated how the mass flow rate of the water contained in the stream water (bottom stream of D, waste water stream), f(water), is calculated. In this calculation, the water contents of P(1) and P(2), both being less than 1000 weight-ppm, are neglected.

f(water)=f_{H2O}(H(1))+f_{H2O}(H(2))+f_{H2O}(M)−f_{H2O}(waste gas) 2.5
f_{H2O}(H(1)) is the mass flow rate of water contained in the stream H(1) and f_{H2O}(H(2)) is the mass flow rate of water contained in the stream H(2) and f_{H2O}(M) is the mass flow rate of water contained in the stream M:

f_{H2O}(H(1))=[(1−c_{NaOH})*f(H(1))]+[(M_{H2O}/M_{NaOH}*c_{NaOH})*f(H(1))] 2.5.1

| Molecular mass M/concentration c | values | units |
|---|---|---|
| M_{H2O} | 18 | kg/kmol |
| M_{NaOH} | 40 | kg/kmol |
| c_{NaOH} in H(1) | 0.5 | kg/kg |

f_{H2O}(H(2))=[(1−c_{KOH})*f(H(2))]+[(M_{H2O}/M_{KOH}*c_{KOH})*f(H(2))] 2.5.2

| Molecular mass M/concentration c | values | units |
|---|---|---|
| M_{H2O} | 18 | kg/kmol |
| M_{KOH} | 56 | kg/kmol |
| c_{KOH} in H(2) | 0.48 | kg/kg |

f_{H2O}(M)=0.001*f(M) 2.5.3

f_{H2O}(waste gas)=0(neglected) 2.5.4

### DESCRIPTION OF THE FIGURES

FIG. 1 shows a schematic overview of a process according to the present invention wherein the rectification column D is operated without top vapor recompression. In particular, a fresh methanol stream M is fed into the upper part of a rectification column D. From the top of the rectification column D, a gas stream T(2), a dry methanol stream, is removed which as passed through the condenser V(4), from which condenser V(4) a waste gas stream T(2w), essentially consisting of inerts and methanol, and a liquid stream T(3) are obtained. The liquid stream T(3) is fed back to the top of the column D. A part of the bottoms stream removed from the column D is fed into the bottom reboiler V(3) of D, the other part of the bottoms stream, essentially consisting of water, is disposed. Further, from the top of the column D, a dry methanol gas stream G is removed, in addition to T(2). This gas stream G, exhibiting a flow rate f(G), is divided into two streams G(1) and G(2), both having the same composition as G. The stream G(1) exhibits a flow rate f(G(1)), the stream G(2) exhibits a flow rate f(G(2)), wherein f(G(1))+f((G2))=f(G). The stream G(1) is then passed through a compressor C(1), and the thus compressed stream G(1) is then fed into the lower part of reactive distillation column K(1), wherein into the upper part of K(1), a liquid aqueous stream H(1) comprising a dissolved alkali metal hydroxide A(1)OH is fed. A part of the bottoms stream removed from the column K(1) is fed into the reboiler V(1) of K(1), the other part of the bottoms stream is the mixture P(1) comprising alkali metal methoxide A(1)OMe and methanol. From the top of the column K(1), which is operated without reflux, a gas stream W(1) essentially consisting of methanol and water is removed, wherein W(1) is fed into a lower part of the rectification column D. The stream G(2) is then passed through a compressor C(2), and the thus compressed stream G(2) is then fed into the lower part of reactive distillation column K(2), wherein into the upper part of K(2), a liquid aqueous stream H(2) comprising a dissolved alkali metal hydroxide A(2)OH is fed. A part of the bottoms stream removed from the column K(2) is fed into the reboiler V(2) of K(2), the other part of the bottoms stream is the mixture P(2) comprising alkali metal methoxide A(2)OMe and methanol. From the top of the column K(2), which is operated without reflux, a gas stream W(2) essentially consisting of methanol and water is removed, wherein W(2) is fed, together with W(1), into a lower part of the rectification column D.

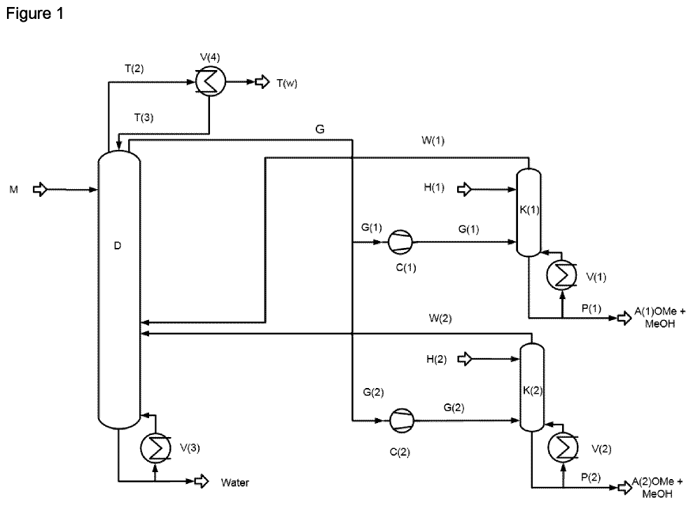

FIG. 2 shows the schematic overview according to FIG. 1, wherein in the top of the reactive distillation columns K(1) and K(2), droplet separating devices D(1) and D(2), preferably demisters, are located, with the streams M(1) and M(2) used for an at least temporary spraying.

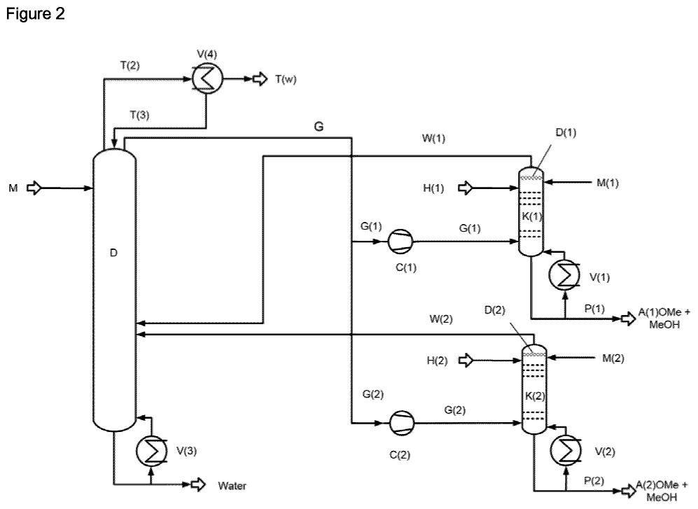

FIG. 3 shows the schematic overview according to FIG. 2, wherein the compressors C(1) and C(2) are not located upstream of K(1) and K(2) for compressing G(1) and G(2), but located downstream of K(1) and K(2) for compressing W(1) and W(2) prior to feeding W(1) and W(2) into D.

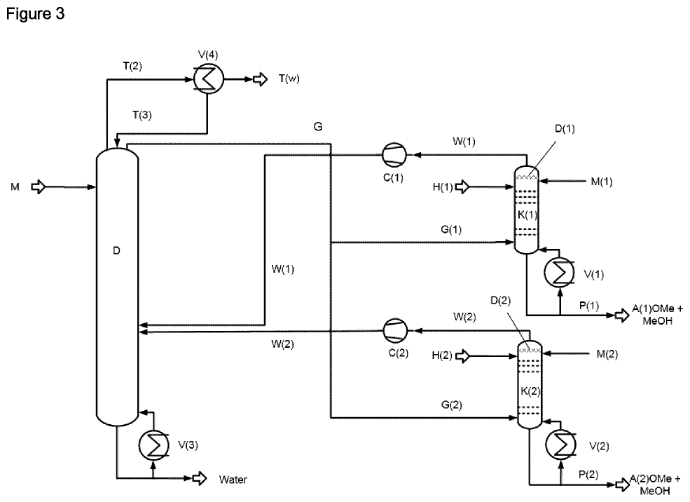

FIG. 4 shows a schematic overview of a process according to the present invention wherein, compared to the process shown in FIG. 1, the rectification column D is operated with top vapor recompression. Regarding said top vapor recompression, a dry methanol top stream is removed from the rectification column D, in addition to G and T(2). This gas stream T(1) is passed through compressor C(3), and this compressed stream T(1) is then passed through the intermediate reboiler V(6) of the column D. The thus obtained compressed and condensed stream T(1) obtained from V(6) is then passed to a first condensate drum. Inerts which are comprised in T(1) are removed as gas stream T(1g) from the first condensate drum via a control valve (not shown) and fed into the condenser V(4) into which also the top stream T(2) is fed. The other part of T(1), T(11), is depressurized into a second condensate drum. Into said second condensate drum, a further stream T(2gl) is fed which is obtained from the condenser V(5) into which the stream T(2g) obtained from V(4), T(2l) is fed. Further, the stream T(2l) obtained from V(4) is fed in to the second condensate drum. From the second condensate drum, a liquid stream T(3) is removed and fed to the top of the column D.

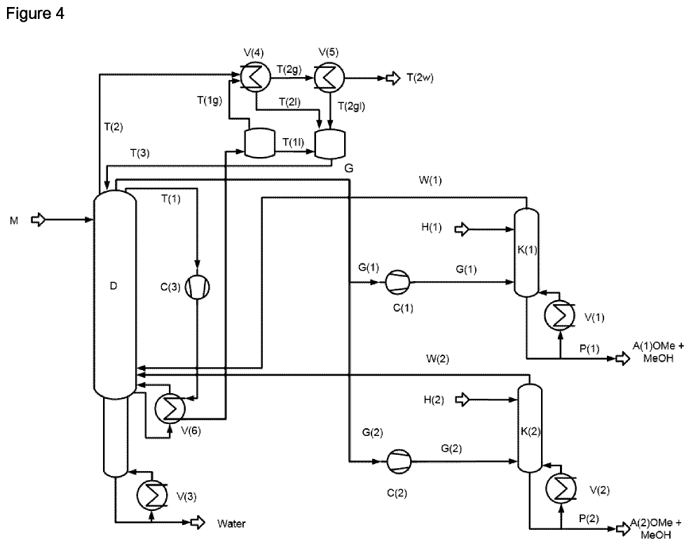

FIG. 5 shows the schematic overview according to FIG. 4, wherein in the top of the reactive distillation columns K(1) and K(2), droplet separating devices D(1) and D(2), preferably demisters, are located, with the streams M(1) and M(2) used for an at least temporary spraying.

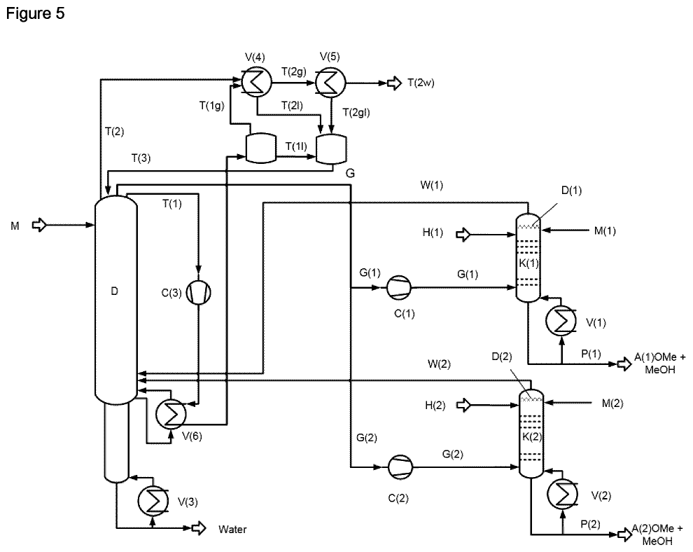

FIG. 6 shows the schematic overview according to FIG. 5, wherein the compressors C(1) and C(2) are not located upstream of K(1) and K(2) for compressing G(1) and G(2), but located downstream of K(1) and K(2) for compressing W(1) and W(2) prior to feeding W(1) and W(2) into D.

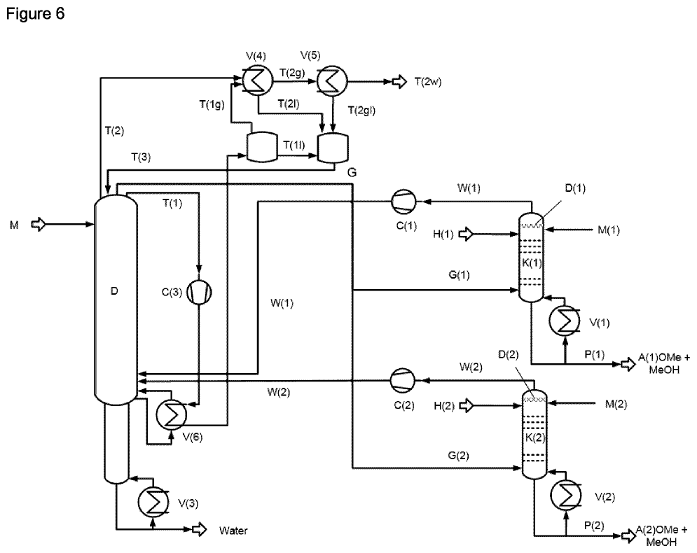

### CITED LITERATURE

US 2002/0183566 A1
US 2008/0296786 A1
WO 2013/168113 A1

1.-15. (canceled)

16. An integrated process for simultaneously preparing n mixtures P(i) comprising alkali metal methoxide and methanol, comprising
providing n reactive distillation columns K(i);
providing n aqueous liquid streams H(i), a given stream H(i) comprising a dissolved alkali metal hydroxide A(i)OH, wherein n is an integer with n≥2 and i=1 . . . n; and
providing a rectification column D;
the process further comprising
(a) providing a stream G comprising methanol;
(b) dividing the stream G into n streams G(i), each stream G(i) having the same composition as G;
(c) preparing the one or more alkali metal methoxides comprising
feeding each stream G(i) into the lower part of a respective reactive distillation column K(i), and feeding the aqueous liquid stream H(i) comprising the dissolved alkali metal hydroxide A(i)OH into the upper part of said reactive distillation column K(i); and
subjecting G(i) and H(i) in each K(i) to reactive distillation conditions, obtaining n top streams W(i) comprising methanol and water; and obtaining n bottoms streams P(i) comprising alkali metal methoxide A(i)OMe and methanol;
(d) feeding each stream W(i) into the lower part of the rectification column D, and feeding a stream M comprising methanol into the rectification column D; and subjecting the n streams W(i) and M in D to distillation conditions, obtaining the stream G according to (a) as a top stream.

17. The process of claim 16, wherein n is in the range of from 2 to 10.

18. The process of claim 16, wherein each alkali metal hydroxide A(i)OH is selected from the group consisting of lithium hydroxide, sodium hydroxide and potassium hydroxide, and wherein a given aqueous liquid stream H(i) comprises an alkali metal hydroxide A(i)OH dissolved in water, in methanol, or in a mixture comprising water and methanol.

19. The process of claim 16, being an integrated process for simultaneously preparing at least 2 mixtures P(i), wherein the mixture P(1) comprises A(1)OMe, and methanol and the mixture P(2) comprises A(2)OMe, and methanol, the process comprising
(a) providing a stream G comprising methanol;
(b) dividing the stream G into at least two streams G(1) and G(2), G(1) and G(2) having the same composition as G;
(c.1) preparing A(1)OMe, comprising
(c.1.1) feeding the stream G(1) into the lower part of a reactive distillation column K(1), and feeding an aqueous liquid stream H(1) comprising dissolved A(1)OH, into the upper part of the reactive distillation column K(1);
(c.1.2) subjecting G(1) and H(1) in K(1) to reactive distillation conditions, obtaining a top stream W(1) comprising methanol and water; and obtaining a bottoms stream P(1) comprising A(1)OMe, and methanol;
(c.2) preparing A(2)OMe, comprising
(c.2.1) feeding the stream G(2) into the lower part of a reactive distillation column K(2), K(2) being arranged in parallel with K(1), and feeding an aqueous liquid stream H(2) comprising dissolved A(2)OH, into the upper part of the reactive distillation column K(2);
(c.2.2) subjecting G(2) and H(2) in K(2) to reactive distillation conditions, obtaining a top stream W(2) comprising methanol and water; and obtaining a bottoms stream P(2) comprising A(2)OMe, and methanol;
(d.1) feeding W(1) and W(2) into the lower part of a rectification column D, and feeding a stream M comprising methanol into the rectification column D;
(d.2) subjecting W(1), W(2) and M in D to distillation conditions, obtaining the stream G according to (a) as a top stream.

20. The process of claim 19, wherein W(1) and W(2) are fed as gas streams into the rectification column D.

21. The process of claim 16, wherein from 99 to 100 weight-% of the stream M consist of methanol and optionally water, wherein the amount of water comprised in the stream M is at most 2000 weight-ppm, wherein the stream M is fed to the upper part of D, at a temperature of M in the range of from ambient temperature to the boiling point of methanol at the column pressure of D.

22. The process of claim 16, wherein the rectification column D is operated at a reflux ratio of at least 0.5:1, wherein for realizing the reflux ratio, the process comprises
(i) removing, in addition to G, a top stream T(2) from the rectification column D, passing said stream T(2) through a condenser V(4), obtaining a liquid stream and a waste gas stream T(2w);
(ii) removing, in addition to G and T(2), a further top stream T(1) from the rectification column D, passing said stream T(2) through a compressor C(3), passing the thus compressed stream through a reboiler V(6), obtaining a liquid stream, wherein the reboiler V(6) is a reboiler of the rectification column D;
(iii) feeding the liquid streams obtained according to (i) and (ii) into the top of the rectification column D;
or
(i) removing, in addition to G, a top stream T(2) from the rectification column D, passing said stream T(2) through a condenser V(4), obtaining a liquid stream T(2l) and a gas stream T(2g); passing the gas stream T(2g) through a condenser V(5), obtaining a liquid stream T(2gl) and a waste gas stream T(2w); and combining the liquid streams T(2l) and (T2gl), obtaining a combined liquid stream T(2cl);
(ii) removing, in addition to G and T(2), a further top stream T(1) from the rectification column D, passing said stream T(2) through a compressor C(3), passing the thus compressed stream through a reboiler V(6), obtaining a liquid stream, wherein the reboiler V(6) is a reboiler of the rectification column D;
(iii) feeding the combined liquid stream obtained according to (i) and the liquid stream obtained according to (ii) into the top of the rectification column D.

23. The process of claim 16, wherein the stream G provided according to (a) by obtaining from distillation according to (d.2) comprises methanol and water, and wherein the water content of G is at most 200 weight-ppm.

24. The process of claim 16, wherein according to (b), the stream G is divided into the two streams G(1) and G(2).

25. The process of claim 16, wherein prior to be fed into the reactive distillation column K(1), the stream G(1) is passed through a compressor C(1), thereby realizing a pressure increase of G(1) in the range of from 0.1 to 0.8 bar(abs);
and/or,
prior to be fed into the reactive distillation column K(2), the stream G(2) is passed through a compressor C(2), thereby realizing a pressure increase of G(2) in the range of from 0.1 to 0.8 bar(abs).

26. The process of claim 16, wherein prior to being fed into the rectification column D, the stream W(1) is passed through a compressor C(1), thereby realizing a pressure increase of W(1) in the range of from 0.1 to 0.8 bar(abs);
and/or,
prior to being fed into the rectification column D, the stream W(2) is passed through a compressor C(2), thereby realizing a pressure increase of W(2) in the range of from 0.1 to 0.8 bar(abs).

27. A chemical production unit for carrying out the process according to claim 16, comprising
a rectification column D comprising
inlet means for feeding the stream M into D;
in a lower part, inlet means for feeding the streams W(i) or one or more combined stream thereof into D;
outlet means for removing the streams T(2) and G or a combined stream thereof from the top of D;
at least one condenser V(4) and optionally a further condenser V(5) arranged downstream of V(4), having inlet means for receiving the stream T(2) and having outlet means for removing the condensed stream T(3) and for removing a waste gas stream;
inlet means for feeding the stream T(3) to the top of D;
a bottom reboiler;
a stream dividing device S for dividing the stream G into n streams G(i);
means for passing the stream G to said stream dividing device S;
n reactive distillation columns K(i), n≥2 and i=1 . . . n; said reactive distillation columns K(i) being arranged in parallel, each reactive distillation column K(i) comprising
in an upper part, an inlet means for feeding a stream H(i) into K(i);
in a lower part, inlet means for feeding a stream G(i) into K(i);
outlet means for removing a stream W(i) from the top of K(i);
a bottom reboiler;
outlet means for removing a bottoms stream from K(i);
a stream dividing means for separating a stream P(i) from the bottoms stream removed from K(i);
means for passing the streams G(i) to the reactive distillation columns K(i);
means for passing the streams W(i) to the rectification column D;
one or more compressors C(i) for compressing either the stream G and/or the streams G(i) and/or the streams W(i).

28. The unit of claim 27, comprising n compressors C(i) arranged upstream of K(i) for compressing the streams G(i) or comprising n compressors C(i) arranged downstream of K(i) and upstream of D for compressing the streams W(i).

29. The unit of claim 27, wherein the inlet means for feeding the streams W(i) or one or more combined stream thereof into D is/are located at a position between the bottoms and the 15^{th} theoretical stage; and/or
wherein the inlet means for feeding the stream M into D are located in the upper part of D.

30. A method comprising providing the chemical production unit according to claim 27 or and simultaneously producing n mixtures P(i) comprising alkali metal methoxide A(i)OMe and methanol, n being an integer with n≥2 and i=1 . . . n, wherein
either at least 2 of the mixtures P(i) comprise different alkali metal methoxides A(i)OMe, and/or at least 2 of the mixtures P(i) comprise the same alkali metal alkoxide A(i)OMe at different concentrations.
